(19) United States
(12) Patent Application Publication
(10) Pub. No.: US 2025/0090589 A1
(43) Pub. Date: Mar. 20, 2025

(54) METHOD OF TREATING PROGRESSIVE HEART FAILURE IN SUBJECTS AT HIGH RISK OF POOR OUTCOMES

(71) Applicant: MESOBLAST INTERNATIONAL SÁRL, Meyrin (CH)

(72) Inventors: Silviu ITESCU, Melbourne (AU); Kenneth BOROW

(21) Appl. No.: 18/711,167

(22) PCT Filed: Nov. 17, 2022
(86) PCT No.: PCT/US2022/080089

§ 371 Date: May 17, 2024

Related U.S. Application Data
(60) Provisional application No. 63/384,200, filed on Nov. 17, 2022.

(30) Foreign Application Priority Data
Nov. 17, 2021 (AU) 2021903706

(51) Int. Cl. A61K 35/28; A61K 9/00; A61K 47/02; A61K 47/12; A61K 47/20; A61K 47/42; A61P 9/04

(52) U.S. Cl. CPC A61K 35/28; A61K 9/0019; A61K 47/02; A61K 47/12; A61K 47/20; A61K 47/42; A61P 9/04

(57) ABSTRACT

The present disclosure relates to methods for treating and/or preventing progressive heart failure in subjects at high risk of poor outcomes. Such methods may be used for treating or preventing progressive heart failure in subjects with micro-vascular disease and/or macro-vascular disease.

### CROSS REFERENCE TO RELATED APPLICATIONS

This application is the U.S. National Phase entry of International Application No. PCT/US2022/080089, filed Nov. 17, 2022, which claims the priority benefit of Australian Application No. 2021903706, filed on Nov. 17, 2021, and U.S. Application No. 63/384,200, filed Nov. 17, 2022, each of which is herein incorporated by reference in its entirety.

### TECHNICAL FIELD

The present disclosure relates to methods for treating and/or preventing progressive heart failure in subjects at high risk of poor outcomes.

### BACKGROUND

Myocardial infarction (MI) is still one of the main causes of mortality and morbidity in developed countries. An update of US Medicare records was published that evaluated data involving 350,509 acute MI hospitalization in patients >65 years who were discharged alive after their event (Schuster et al. (2004) Physiol Heart Circa Physiol., 287 (2): 525-32). Within the first year post the index event, 25.9% of the MI patients died with 50.5% re-hospitalized. In the month after a MI, the likelihood of death was 21 times higher and the likelihood of hospitalization and was 12 times higher than among the general Medicare-age population.

During the past decade, numerous clinical trials evaluating novel drug therapies have been conducted in patients with advanced heart failure (HF). Despite progress made in reducing morbidity and mortality in patients with HF, those with advanced disease continue to experience an unfavourable clinical course characterized by frequent hospitalizations and premature death.

Clearly, there is a need in the art for treating or preventing progressive heart failure.

### SUMMARY

The present inventors have surprisingly found that cell therapy is particularly effective in certain subjects with progressive heart failure, in particular subjects with poor outcomes such as subjects with micro-vascular disease and/or macro-vascular disease. For example, cell therapy is particularly effective in subjects with progressive heart failure in addition to myocardial ischemia and/or diabetes. Accordingly, in an example, the present disclosure relates to a method for treating or preventing progressive heart failure in a subject, the method comprising administering to the subject a composition comprising mesenchymal lineage precursor or stem cells, wherein the subject has micro-vascular disease and/or macro-vascular disease.

In another example, the present disclosure relates to a method of reducing risk of cardiac death, non-fatal myocardial infarction or non-fatal stroke in a subject, the method comprising administering to the subject a composition comprising mesenchymal lineage precursor or stem cells, wherein the subject has micro-vascular disease and/or macro-vascular disease. In an example, the method reduces non-fatal risk of non-fatal events such as subsequent MI or stroke. Accordingly, in another example, the present disclosure relates to a method of reducing risk of myocardial infarction or stroke in a subject, the method comprising administering to the subject a composition comprising mesenchymal lineage precursor or stem cells, wherein the subject has micro-vascular disease and/or macro-vascular disease.

In another example, the present disclosure relates to a method of selecting heart failure patients for treatment with cell therapy, the method comprising i) assessing the subject for a micro-vascular disease and/or a macro-vascular disease, and ii) selecting a subject having a micro-vascular disease and/or a macro-vascular disease for treatment, preferably, wherein the treatment comprises administering a composition comprising mesenchymal lineage precursor or stem cells.

In these example, the micro-vascular disease and/or a macro-vascular disease is myocardial ischemia and/or diabetes.

In an example, the subject's CRP level is ≥2 mg/L. In another example, the subject's CRP level is <5 mg/L. In another example, the subject's CRP level is <4 mg/L. In another example, the subject's CRP level is <3 mg/L. In another example, the subject's CRP level is between 2 and 5 mg/L. In another example, the subject's CRP level is between 2 and 4 mg/L. In another example, the subject's CRP level is between 2 and 3 mg/L.

In another example, the subject has a LVEF of less than about 45%. In another example, the subject has an LVEF less than 40%. In an example, LVEF is measured by two-dimensional echocardiogram. In an example, the subjects LVEF is ≤35%, wherein LVEF is measured by multi-gated acquisition scan.

In an example, the subject has a LVESV greater than 70 ml. In another example, the subject has a LVESV greater than 100 ml. In another example, the subject has a LVESV greater than 130 ml. In another example, the subject has a LVESV between 70 ml and 160 ml. In an example, the subject has Class II heart failure according to the New York Heart Association (NYHA) classification scale.

In another example, the subject's level of N-terminal pro-B-type natriuretic peptide (NT-proBNP) is:
>1000 pg/mL, or,
between 1000 pg/ml and 2500 pg/ml.

In an example, the subject has had a heart failure hospitalisation event over the previous 9 months. In another example, the subject has had at least 1 outpatient urgent care HF visit requiring intravenous diuretic, vasodilator. In another example, the subject has had positive inotropic therapy between 1 and 9 months before treatment.

In an example, the subject has persistent left ventricular dysfunction.

In an example, the subject has a reduced risk of cardiac death after treatment. In this example, the reduced risk may be relative to risk of cardiac death in a subject that has not been administered mesenchymal lineage precursor or stem cells.

In another example, the subject has a reduced risk of ischaemic MACE (MI or stroke) after treatment.

In an example, the composition is administered transendocardially and/or intravenously. For example, the composition may be administered transendocardially.

In an example, the mesenchymal lineage precursor or stem cells are mesenchymal precursor cells (MPCs). In an example, the MPCs are isolated from bone mononuclear cells with an anti-STRO-3 antibody.

In another example, the present disclosure relates to a method for treating or preventing progressive heart failure in a subject, the method comprising administering to the subject a composition comprising cells. In an example, the subject has Class II or Class III heart failure according to the New York Heart Association (NYHA) classification scale. In an example, the subject may have less than Class III heart failure according to the New York Heart Association (NYHA) classification scale. In an example, the subject has Class II heart failure according to the New York Heart Association (NYHA) classification scale. Accordingly, in an example, the present disclosure relates to a method for treating or preventing progressive heart failure in a subject, the method comprising administering to the subject a composition comprising cells, wherein the subject has Class II heart failure according to the New York Heart Association (NYHA) classification scale.

In another example, the present disclosure relates to a method of reducing progression of heart failure in a subject, the method comprising administering to the subject a composition comprising cells, wherein the subject has Class II heart failure according to the New York Heart Association (NYHA) classification scale.

In another example, the present disclosure relates to a method of reducing cardiac death in a subject with Class II heart failure according to the New York Heart Association (NYHA) classification scale, the method comprising administering to the subject a composition comprising cells.

In another example, the present disclosure relates to a method of selecting heart failure patients for treatment with cell therapy, the method comprising i) assessing heart failure according to the New York Heart Association (NYHA) classification scale, and ii) selecting a subject having Class II heart failure according to NYHA. In an example, the method further comprises administering a composition comprising cells.

In an example, the cells induce new blood vessel formation in target tissue. In an example, the cells promote arteriogenesis. In an example, the cells secrete factors that protect at risk myocardium. Accordingly, in an example, the present disclosure relates to a method for treating or preventing progressive heart failure in a subject, the method comprising administering to the subject a composition comprising cells, wherein the subject has Class II heart failure according to the New York Heart Association (NYHA) classification scale, and wherein the cells induce new blood vessel formation in target tissue and/or secrete factors that protect at risk myocardium.

In an example, the cells are mesenchymal lineage precursor or stem cells (MLPSCs). In an example, the MLPSCs are STRO-1+. In an example, the MLPSCs are mesenchymal stem cells (MSCs). In an example, the MLPSCs are allogeneic. In an example, the cells are culture expanded. In this example, the cells may be TNAP+ before they are culture expanded. In an example, the cells have been cryopreserved.

In another example, the methods of the disclosure comprise the steps of: i) selecting a subject having Class II heart failure according to the New York Heart Association (NYHA) classification scale, and ii) administering to the subject a composition comprising cells which induce new blood vessel formation in target tissue.

In another example, administering the composition inhibits the subject's progression to NYHA class III progressive heart failure.

In an example, the subject's level of N-terminal pro-B-type natriuretic peptide (NT-proBNP) is less than 2200 pg/ml. In another example, the subject's NT-proBNP is less than 2000 pg/ml, prior to administering the cells. In another example, the subject's level of NT-proBNP is between 1000 pg/ml and 2000 pg/ml prior to administering the cells.

The present inventors have also surprisingly found that cell therapy is particularly effective in subjects with earlier stages of progressive heart failure that have elevated C-reactive protein (CRP) levels. Accordingly, in an example, the subject's CRP level is elevated. In an example, the subject's CRP level is >1 mg/L. In an example, the subject's CRP level is >1.5 mg/L. In an example, the subject's CRP level is ≥2 mg/L. In an example, the subject's CRP level is >2 mg/L. In another example, the subject's CRP level is between 1.5 and 5 mg/L. In another example, the subject's C-reactive protein (CRP) level is <5 mg/L, preferably <4 mg/L. In another example, the subject's CRP level is between 1 and 5 mg/L. In another example, the subject's CRP level is between 1.5 and 5 mg/L.

In another example, the subject has had a heart failure hospitalisation event over the previous 9 months.

In another example, the subject has a LVEF of less than about 45%, preferably less than 40%. In another example, the subject has persistent left ventricular dysfunction.

In another example, the subject's heart failure results from an ischaemic event.

In another example, the subject's heart failure results from a non-ischaemic event.

In an example, the subject has a reduced risk of cardiac death after treatment. In an example, the reduced risk is relative to risk of cardiac death in a subject with NYHA class III progressive heart failure. In another example, the subject has a reduced risk of ischaemic MACE (MI or stroke) after treatment.

In an example, a class III heart failure subject has a reduced risk of ischaemic MACE (non-fatal MI or non-fatal stroke). In another example, a class III heart failure subject has a reduced risk of cardiac death after treatment. In another example, a class III heart failure subject has a reduced risk of ischaemic MACE and cardiac death after treatment. In an example, the class III heart failure subjects CRP level is ≥2 mg/L.

In an example, the composition is administered transendocardially and/or intravenously. In an example, the composition is administered transendocardially.

The present inventors also surprisingly identified that cell therapy reduced the risk of ischemic events in subjects with cardiomyopathy. Accordingly, in an example, the present disclosure also encompasses a method of reducing risk of cardiac death or a non-fatal ischemic event in a subject, the method comprising administering to the subject a composition comprising cells. In an example, the subject has a cardiomyopathy. In another example, the subject has micro-vascular disease and/or macro-vascular disease. For example, the subject may have myocardial ischemia and/or diabetes. In an example, the subject has myocardial ischemia. In another example, the subject has diabetes.

In an example, the ischemic event is formation of a cerebrovascular or cardiac occlusion. In an example, the ischemic event is a stroke or myocardial infarction. In an example, the subject has non-ischemic cardiomyopathy. In an example, the cells are administered transendocardially. In an example, the subject has Class II or Class III heart failure according to the New York Heart Association (NYHA) classification scale. In an example, the subject has active inflammation. In an example, the subject has Class II heart failure and “active inflammation”. In an example, the active inflammation is characterised by a level of CRP >1.5 mg/L. In another example, active inflammation is characterised by a level of CRP >2 mg/L. Exemplary cells are discussed above and throughout the present disclosure. In an example, the cells induce new blood vessel formation in target tissue. In an example, the cells promote arteriogenesis. In an example, the cells secrete factors that protect at risk myocardium. In an example, the cells are MLPSCs. In an example, the MLPSCs are STRO-1+. In an example, the MLPSCs are mesenchymal stem cells (MSCs). In an example, the MLPSCs are allogeneic. In an example, the cells are culture expanded. In this example, the cells may be TNAP+ before they are culture expanded. In an example, the cells have been cryopreserved.

In an example, the subject's level of N-terminal pro-B-type natriuretic peptide (NT-proBNP) is between 1000 pg/ml and 2000 pg/ml prior to administering the cells. In another example, the subject's C-reactive protein (CRP) level is elevated. In another example, the subject's CRP level is ≥1 mg/L. In another example, the subject's CRP level is ≥2 mg/L. In another example, the subject's CRP level is between 2 and 5 mg/L. In another example, the subject's CRP level is between 3 and 5 mg/L.

In an embodiment of the above examples, the methods of the disclosure comprise administering between 1×10⁷ and 2×10⁸ cells.

In another example, the administered composition further comprises Plasma-Lyte A, dimethyl sulfoxide (DMSO), human serum albumin (HSA). In an example, the administered composition further comprises Plasma-Lyte A (70%), DMSO (10%), HSA (25%) solution, the HSA solution comprising 5% HSA and 15% buffer. In an example, the composition comprises greater than 6.68×10⁶ viable cells/mL.

In another example, the composition comprises human bone marrow-derived allogeneic mesenchymal precursor cells (MPCs) isolated from bone mononuclear cells with anti-STRO-3 antibodies, expanded ex vivo, and cryopreserved.

In an example, the ischemic events are one or more of heart attack, stroke or cardiac death. In an example, the method reduces risk of 3-point MACE.

The present inventors have also surprisingly identified that elevated CRP levels is associated with increased cardiac death risk myocardial infarction or stroke. Accordingly, in an example, the present disclosure relates to a method for determining elevated risk of one or more of cardiac death, myocardial infarction or stroke in a subject, the method comprising measuring the level of CRP in a sample obtained from a subject, wherein elevated CRP indicates elevated risk of cardiac death, myocardial infarction or stroke. In an example, the subject has progressive heart failure. In an example, the subject's heart failure is NYHA Class II heart failure. In another example, a level of CRP >1 mg/L indicates elevated risk of cardiac death, myocardial infarction or stroke. In another example, a level of CRP >1.5 mg/L indicates elevated risk of cardiac death, myocardial infarction or stroke. In another example, a level of CRP ≥2 mg/L indicates elevated risk of cardiac death, myocardial infarction or stroke. In an example, the method determines elevated risk of cardiac death.

### BRIEF DESCRIPTION OF DRAWINGS

FIG. 1: Reduced Incidence of Ischemic MACE (MI, Stroke).

FIG. 2: Reduced Incidence of Ischemic MACE (MI, Stroke); NYHA Class II and Class III.

FIG. 3: Reduced Incidence of Ischemic MACE (MI, Stroke); ischemic and non-ischemic.

FIG. 4: Cardiac death in all treated (n=537); Class II (n=206); Class III patients (n=331).

FIG. 5: Cardiac death in NYHA Class II patients; ischemic and non-ischemic.

FIG. 6: Cardiac death in trial patients.

FIG. 7: A) TTFE composite IMM MACE; B) Rate normalised composite IMM MACE; C) Curves for all treated patients with baseline CRP ≥2 mg/L vs CRP ≤2 mg/ml. Curves shown for Non-fatal MI or Non-fatal stoke; 3-point TTFE composite IMM MACE for CV death or non-fatal MI or non-fatal stroke.

FIG. 8: A) TTFE Irreversible morbidity; B) Rate normalised irreversible morbidity; C) Curves for irreversible morbidity TTFE MACE (non-fatal MI or non-fatal stroke) for All treated (n=537); Class II (n=206); Class III patients (n=331).

FIG. 9: Reduced Incidence of composite cardiac death or Ischemic MACE (MI, Stroke); All treated (n=537); Class II (n=206); Class III patients (n=331).

FIG. 10. Cardiac Death in NYHA Class II patients-Multiple Years of Follow-up.

FIG. 11. NYHA Class II patients with baseline hsCRP ≥2 mg/L are at significantly greater risk of progression to cardiac death.

FIG. 12. NYHA Class II patients with baseline hsCRP ≥2 mg/L are at significantly greater risk of 3-point MACE (Cardiac death/MI/stroke).

FIG. 13. Heart failure patients with myocardial ischemia and/or diabetes have worse outcomes, as measured by 3-Point IMM MACE, than other heart failure patients.

FIG. 14. Cell therapy reduced risk of 3-Point TTFE composite IMM MACE in high-risk patients with myocardial ischemia &/or diabetes by 36%.

FIG. 15. Composite 3-point MACE and inflammation: In patients with myocardial ischemia and/or diabetes with hsCRP>2 mg/L cell therapy reduced risk of TTFE for 3-Point MACE by 54%.

FIG. 16. High Risk Of 3-Point composite MACE (MI, Stroke or CV Death) in ischemic CHF Patients: Cell therapy protects against 3-Point MACE in these subjects with CRP levels ≥2 mg/L.

FIG. 17. High Risk Of non-fatal MACE (MI or Stroke) in all diabetic CHF Patients: Cell therapy protects against non-fatal MACE (MI or Stroke) in diabetic CHF patients with CRP levels ≥2 mg/L.

FIG. 18. High Risk Of 3-Point composite MACE (MI, Stroke or CV Death) in diabetic CHF Patients: Cell therapy protects against 3-Point MACE in these subjects with CRP levels ≥2 mg/L.

### DETAILED DESCRIPTION

### General Techniques and Definitions

Unless specifically defined otherwise, all technical and scientific terms used herein shall be taken to have the same meaning as commonly understood by one of ordinary skill in the art (e.g., in cell culture, molecular biology, stem cell culture, immunology, and biochemistry).

Unless otherwise indicated, cell culture techniques and assays utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein, the term “about”, unless stated to the contrary, refers to +/−10%, more preferably +/−5%, of the designated value.

The terms “level” and “amount” are used to define the amount of a particular substance in a sample from a subject or in a cell culture media (or sample therefrom). For example, a particular concentration, weight, percentage (e.g. v/v %) or ratio can be used to define the level of a particular substance in a sample. In an example, the level is expressed in terms of how much of a particular marker is expressed by cells of the disclosure under culture conditions. In an example, expression represents cell surface expression. In another example, the level is expressed in terms of how much of a particular marker is release from cells described herein under culture conditions. In an example, the sample is obtained from a patient or subject (e.g. a blood sample) and the level of a substance is measured in the sample to determine the level of the substance in the sample.

In an example, the level is expressed in pg/ml. For example, the level of NT-proBNP can be expressed in pg/ml. In an example, the level is expressed in mg/L. For example, the level of CRP can be expressed in mg/L. In another example, the level is expressed in pg per 10⁶ cells.

In an example, the level of a particular marker in a cell culture medium is determined under culture conditions. The term “culture conditions” is used to refer to cells growing in culture. In an example, culture conditions refers to an actively dividing population of cells. Such cells may, in an example, in exponential growth phase. For example, the level of a particular marker can be determined by taking a sample of cell culture media and measuring the level of marker in the sample. In another example, the level of a particular marker can be determined by taking a sample of cells and measuring the level of the marker in the cell lysate. Those of skill in the art that secreted markers will be measured by sampling the culture media while markers expressed on the surface of the cell may be measured by assessing a sample of cell lysate. In an example, the sample is taken when the cells are in exponential growth phase. In an example, the sample is taken after at least two days in culture.

Culture expanding cells from a cryopreserved intermediate means thawing cells subject to cryogenic freezing and in vitro culturing under conditions suitable for growth of the cells.

In an example, the “level” or “amount” of a particular marker is determined after cells have been cryopreserved and then seeded back into culture. For example, the level is determined after a first cryopreservation of cells. In another example, the level is determined after a second cryopreservation of cells. For example, cells may be culture expanded from a cryopreserved intermediate, cryopreserved a second time before being re-seeded in culture so that the level of a particular marker can be determined under culture conditions.

As used herein, the terms “treating”, “treat”, “treatment”, “reducing progression” include administering a population of mesenchymal lineage stem or precursor cells and/or progeny thereof and/or soluble factors derived therefrom and/or extracellular vesicles derived therefrom to thereby reduce or eliminate at least one symptom of progressive heart failure or, in the context of reducing progression, delay development of the same.

In an example, the present disclosure encompasses selecting certain subjects with progressive heart failure for treatment with a cellular composition disclosed herein. In an example, subjects with Class II heart failure are selected for treatment. In another example, subjects with active inflammation are selected for treatment. For example, subjects with CRP levels ≥2 mg/L are selected for treatment. In another example, subjects with micro-vascular disease and/or macro-vascular disease are selected for treatment. For example, subjects with myocardial ischemia and/or diabetes are selected for treatment. In an example, subjects with active inflammation and either class II heart failure and/or micro-vascular disease and/or macro-vascular disease are selected for treatment.

The term “subject” as used herein refers to a human subject. For example, the subject can be an adult. In another example, the subject can be a child. In another example, the subject can be an adolescent. Terms such as “subject”, “patient” or “individual” are terms that can, in context, be used interchangeably in the present disclosure. Subjects in need of treatment include those already having progressive heart failure as well as those in which progressive heart failure is to be prevented, delayed or halted.

In an example, compositions of the disclosure comprise genetically unmodified mesenchymal precursor lineage or stem cells. As used herein, the term “genetically unmodified” refers to cells that have not been modified by transfection with a nucleic acid. For the avoidance of doubt, in the context of the present disclosure a mesenchymal lineage precursor or stem cell transfected with a nucleic acid encoding a protein would be considered genetically modified.

As used herein, the term “sample” refers to an extract from a subject in which CRP levels can be measured. The “sample” includes extracts and/or derivatives and/or fractions of the sample. In the present disclosure, any biological material can be used as the above-mentioned sample so long as it can be collected from the subject and assayed to determine the level of CRP in the subject. In an example, the sample is a blood sample. In an example, the blood sample is obtained from a subject with NYHA Class II heart failure.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Those skilled in the art will appreciate that the disclosure described herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific embodiments described herein, which are intended for the purpose of exemplification only. Functionally-equivalent products, compositions and methods are clearly within the scope of the disclosure, as described herein.

Any example disclosed herein shall be taken to apply mutatis mutandis to any other example unless specifically stated otherwise.

### Progressive Heart Failure

Cardiomyopathy is a disease of the heart muscle that makes it harder for the heart to pump blood to the rest of the body. When the heart is unable to pump sufficiently to maintain blood flow to meet the needs of the body heart failure can occur. Cardiomyopathy can occur after an ischemic or non-ischemic event. One cause of ischemic heart failure is systolic dysfunction following a myocardial infarction (MI). A MI occurs when blood stops flowing properly to a part of the heart. The lack of blood supply results in a localized area of myocardial necrosis referred to as an infarct or infarction. The infarcted heart is unable to pump sufficiently to maintain blood flow to meet the needs of the body leading to multiple pathophysiologic responses and ultimately heart failure. Non-ischemic cardiomyopathy is not related to known coronary artery disease. One example, is dilated cardiomyopathy (DCM) where the heart's ability to pump blood is decreased because the heart's main pumping chamber, the left ventricle, becomes enlarged, dilated and weak.

Once the heart is unable to pump sufficiently to maintain blood flow to meet the needs of the body, a series of compensatory mechanisms are initiated, serving to buffer the fall in cardiac output and assisting to maintain sufficient blood pressure to perfuse the vital organs. As a result, patients with heart failure may not progress for extended periods of time. However, the compensatory mechanisms eventually fail to compensate for the damaged heart, resulting in a progressive decline in cardiac output, termed “progressive heart failure”. In the context of the present disclosure, the terms chronic heart failure, congestive heart failure, congestive cardiac failure, systolic dysfunction and advanced heart failure can be used interchangeably with “progressive heart failure”.

The methods of the present disclosure can be used to treat progressive heart failure in a specific populations of MI subjects. Subjects in need of treatment include those already having progressive heart failure as well as those in which progressive heart failure is to be prevented, delayed or halted. In these examples, the subject can have Class II or Class III progressive heart failure based on the NYHA. For example, the subject can have Class II progressive heart failure.

In a first example, the present disclosure relates to the treatment of subjects, defined based on the New York Heart Association (NYHA) classification scale. In an example, the subject's progressive heart failure is less than Class III. In an example, the subject has Class II heart failure. In an example, the NYHA classification is assigned based on the subject's symptoms. For example, the NYHA classification can be assigned based on the following Table:

| Class | Patient Symptoms |
|---|---|
| I | No limitation of physical activity. Ordinary physical activity does |
| | not cause undue fatigue, palpitation, dyspnea (shortness of breath). |
| II | Slight limitation of physical activity. Comfortable at rest. |
| | Ordinary physical activity results in fatigue, |
| | palpitation, dyspnea (shortness of breath). |
| III | Marked limitation of physical activity. Comfortable at rest. Less |
| | than ordinary activity causes fatigue, palpitation, or dyspnea. |
| IV | Unable to carry on any physical activity without |
| | discomfort. Symptoms of heart failure at rest. If any |
| | physical activity is undertaken, discomfort increases. |

In an example, the subject's heart failure results from an ischemic event. In an example, the subject's heart failure results from a myocardial infarction (MI). For example, the subject can be a MI subject. The term “myocardial infarction (MI) subject” is used to define subjects who have had a myocardial infarction. In an example, the subject's heart failure results from a non-ischemic cardiomyopathy.

In a second example, the present disclosure relates to the treatment of subjects with progressive heart failure and active inflammation. “Active inflammation” is defined by elevated C-reactive protein levels. In an example, active inflammation is characterised by CRP levels ≥2 mg/L. Accordingly, in this second example, the present disclosure relates to the treatment of subjects with progressive heart failure and CRP levels ≥2 mg/L. In an example, these subjects can have Class II or Class III progressive heart failure based on the NYHA. In another example, these subjects have micro-vascular disease and/or macro-vascular disease. For example, these subject can have ischemia and/or diabetes. Accordingly, in an example, the subject can have progressive heart failure, CRP levels ≥2 mg/L, Class II or Class III progressive heart failure and micro-vascular disease and/or macro-vascular disease. In another example, the subject can have progressive heart failure, CRP levels ≥2 mg/L, Class II or Class III progressive heart failure and ischemia and/or diabetes. In another example, the subject can have progressive heart failure, CRP levels ≥2 mg/L, Class II or Class III progressive heart failure and ischemia. In another example, the subject can have progressive heart failure, CRP levels ≥2 mg/L, Class II or Class III progressive heart failure and diabetes. In another example, the subject can have progressive heart failure, CRP levels ≥2 mg/L, Class II progressive heart failure and ischemia. In another example, the subject can have progressive heart failure, CRP levels ≥2 mg/L, Class II progressive heart failure and diabetes.

“C-reactive protein” or “CRP” is an inflammatory mediator whose levels are raised under conditions of acute inflammatory recurrence and rapidly normalize once the inflammation subsides. In an example, subjects treated according to the present disclosure have elevated risk or cardiac death. In an example, subjects treated according to the present disclosure may have elevated CRP. The term “elevated CRP” is used in the context of the present disclosure to refer to CRP levels that are increased relative to baseline CRP levels. In an example, CRP levels ≥1 mg/L are elevated. In another example, CRP levels ≥1.5 mg/L are elevated. In another example, CRP levels ≥2 mg/L are elevated.

In a third example, the present disclosure relates to the treatment of subjects with progressive heart failure and micro-vascular disease and/or macro-vascular disease. “Microvascular disease” (sometimes called small artery disease or small vessel disease) is heart disease that affects the walls and inner lining of tiny coronary artery blood vessels that branch off from the larger coronary arteries. In coronary MVD, the heart's coronary artery blood vessels may not necessarily have plaque, but may rather have damage to the inner walls of the blood vessels which can lead to spasms and decreased blood flow to the heart muscle. In an example, the microvascular disease is “myocardial ischemia”, a condition characterised by obstructed blood flow to the heart muscle (myocardium) due to a partial or complete blockage of a coronary artery. Examples of myocardial ischemia include ischemic heart failure, angina and stroke. “Macrovascular disease” is characterised by the process of atherosclerosis, which leads to narrowing of arterial walls in the coronary vascular system. Atherosclerosis is thought to result from chronic inflammation and injury to the arterial wall(s) in the coronary vascular system. By driving inflammation and slowing blood flow, diabetes dramatically accelerates atherosclerosis and therefore represents one example of macrovascular disease. In an example, the diabetes is type I or type II diabetes. In an example, the diabetes is type II diabetes.

Accordingly, in an example, the subject can have progressive heart failure and micro-vascular disease and/or macro-vascular disease. In another example, the subject can have progressive heart failure and ischemia and/or diabetes. In another example, the subject can have progressive heart failure and ischemia. In another example, the subject can have progressive heart failure and diabetes. In these examples, the subject may also have active inflammation. For example, the subject can have progressive heart failure, CRP levels ≥2 mg/L and micro-vascular disease and/or macro-vascular disease. In another example, the subject can have progressive heart failure, CRP levels ≥2 mg/L and ischemia and/or diabetes. In another example, the subject can have progressive heart failure, CRP levels ≥2 mg/L and ischemia. In another example, the subject can have progressive heart failure, CRP levels ≥2 mg/L and diabetes.

Subjects from the above referenced first, second and third examples can be further characterised as follows. In an example, subjects treated according to the present disclosure have an initial CRP level ≥2 mg/L. For example, the subject can have Class II or Class III heart failure and an initial CRP level ≥2 mg/L. In another example, the subject can have Class II heart failure and an initial CRP level ≥2 mg/L. In an example, subjects treated according to the present disclosure have an initial CRP level <5 mg/L. In another example, subjects have an initial CRP level <4 mg/L. In another example, subjects have an initial CRP level between 2 and 6 mg/L. In another example, subjects have an initial CRP level between 3 and 6 mg/L. In another example, subjects have an initial CRP level between 4 and 5 mg/L.

There are various assays available for measuring CRP levels such as antibody based immunoassays. For example, CRP levels can be measured in blood samples using an Enzyme-Linked Immunosorbent (ELISA) assay. In an example, a blood sample is obtained from a patient and then purified before being contacted with anti-CRP antibody. Extent of antibody binding is used to quantify the level of CRP in the blood sample (e.g. mg/L).

B-type natriuretic peptide (BNP) is a hormone produced by the heart. N-terminal (NT)-pro hormone BNP (NT-proBNP) is a non-active prohormone that is released from the same molecule that produces BNP. Both BNP and NT-proBNP are released in response to changes in pressure inside the heart. These changes can be related to heart failure and other cardiac problems. Levels goes up when heart failure develops or gets worse, and levels goes down when heart failure is stable. Accordingly, BNP is an effective marker of heart failure progression. In an example, the subject's level of NT-proBNP is less than 2500 pg/ml prior to administering a composition of the disclosure. In another example, the subject's level of NT-proBNP is less than 2400 pg/ml prior to administering a composition of the disclosure. In another example, the subject's level of NT-proBNP is less than 2000 pg/ml prior to administering a composition of the disclosure. In another example, the subject's level of NT-proBNP is less than 1900 pg/ml prior to administering a composition of the disclosure. In another example, the subject's level of NT-proBNP is between 2200 pg/ml and 1000 pg/ml prior to administering a composition of the disclosure. In another example, the subject's level of NT-proBNP is between 2200 pg/ml and 1100 pg/ml prior to administering a composition of the disclosure. In another example, the subject's level of NT-proBNP is between 2100 pg/ml and 1200 pg/ml prior to administering a composition of the disclosure. In another example, the subject's level of NT-proBNP is between 2000 pg/ml and 1500 pg/ml prior to administering a composition of the disclosure.

In another example, the subject has had a heart failure hospitalisation event over the previous 12 months prior to administration of a composition disclosed herein. In another example, the subject has had a heart failure hospitalisation event over the previous 9 months prior to administration of a composition disclosed herein. In another example, the subject has had a heart failure hospitalisation event over the previous 6 to 12 months prior to administration of a composition disclosed herein. In an example, the heart failure hospitalisation event is worsening signs and symptoms of heart failure. In another example, the heart failure hospitalisation event

In another example, the subject is able to walk at least 320 meters in 6 minutes prior to administering a composition of the disclosure. In another example, the subject is able to walk at least 330 meters in 6 minutes prior to administering a composition of the disclosure. In another example, the subject is able to walk at least 340 meters in 6 minutes prior to administering a composition of the disclosure. In another example, the subject is able to walk at least 350 meters in 6 minutes prior to administering a composition of the disclosure.

In an example, subjects can have persistent left ventricular dysfunction. Left ventricular dysfunction is characterised by a decrease in myocardial contractility. A reduction in the left ventricular ejection fraction (LVEF) results when myocardial contractility is decreased within the left ventricle. Thus, LVEF provides one way of determining left ventricular dysfunction. Another parameter of left ventricular function is left ventricular end systolic volume (LVESV), a measurement of the adequacy of cardiac emptying, related to systolic function. LVEF and LVESV are often used together to provide an assessment of left ventricular systolic performance and characterise persistent left ventricular dysfunction.

LVEF and LVESV can be measured by a number of methods known in the art such as echocardiogram (e.g. two dimensional echocardiogram), Single Photon Emission Computed Tomography (SPECT), cardio magnetic resonance imaging (cMRI) or multi-gated acquisition scan.

In an example, a subject having an LVEF of less than 45% has left ventricular dysfunction. In other examples, a subject with a LVEF of less than about 44%, 43%, 42%, 41% has left ventricular dysfunction. In another example, a subject with a LVEF of less than about 40% has left ventricular dysfunction. In other examples, a subject with a LVEF of less than about 39%, 38%, 37%, 36%, 35%, 34%, 33%, 32%, 31%, 30% has left ventricular dysfunction.

In the context of the present disclosure the term “persistent left ventricular dysfunction” is used to define left ventricular dysfunction that persists over a period of time or series of measurements. For example, “persistent left ventricular dysfunction” can include left ventricular dysfunction that persists for between about 1 to about 14 days or longer.

In an example, the subject has a LVEF of less than 45%. In another example, the subject has a LVEF of less than 40%. In other examples, the subject has a LVEF of less than 39%, 38%, 37%, 36%, 35%, 34%, 33%, 32%, 31%, 30%.

In an example, the subject has a LVESV greater than 70 ml. In another example, the subject has a LVESV greater than 100 ml. In another example, the subject has a LVESV greater than 130 ml. In another example, the subject has a LVESV between 70 ml and 160 ml. In these examples, the subject can also have an above referenced LVEF. For example, a subject can have a LVESV greater than 70 ml and a LVEF <45%.

In an example, the subject's heart failure results from an ischaemic event or from a non-ischaemic event. In an example, the subject's heart failure results from an ischaemic event disclosed below.

The methods of the present disclosure relate to the treatment of the progressive decline in cardiac output characteristic of progressive heart failure. Accordingly, “treat” and “treatment”, in the context of the present disclosure refers to both therapeutic treatment and prophylactic or preventative measures.

In an example, treatment includes administering a composition of the disclosure. In an example, methods of the present disclosure reduce or inhibit progression of progressive heart failure. In an example, treatment inhibits the subject's progression to NYHA class III progressive heart failure. In another example, treatment reduces the risk of cardiac death. In an example, the reduced risk of cardiac death is relative to risk of cardiac death in a subject with NYHA class III progressive heart failure. In another example, the risk of ischemic MACE (MI or stroke) is reduced after treatment. In an example, risk of ischemic MACE (MI or stroke) is reduced by at least 50% relative to baseline. In another example, risk of ischemic MACE (MI or stroke) is reduced by at least 55% relative to baseline. In another example, risk of ischemic MACE (MI or stroke) is reduced by at least 60% relative to baseline. In another example, risk of ischemic MACE (MI or stroke) is reduced by at least 65% relative to baseline. In another example, risk of ischemic MACE (MI or stroke) is reduced by at least 70% relative to baseline. In another example, risk of ischemic MACE (MI or stroke) is reduced by at least 50% to 70% relative to baseline.

In another example, the risk of 3-Point MACE (Cardiac death/MI/stroke) is reduced after treatment. In the context of the present disclosure, “3-point MACE” is used to refer to is defined as a composite of cardiovascular death, nonfatal myocardial infarction and nonfatal stroke (Cardiac death/MI/stroke). In an example, risk of 3 point MACE is reduced by at least 30% relative to baseline. In another example, risk of 3 point MACE is reduced by at least 40% relative to baseline. In another example, risk of 3 point MACE is reduced by at least 45% relative to baseline. In another example, risk of 3 point MACE is reduced by at least 50% relative to baseline. In another example, risk of 3 point MACE is reduced by at least 30% to 50% relative to baseline.

In an example, treatment increases patient survival. In an example, treatment increases the probability of a subject surviving for at least 1000 days after initiation of treatment. In another example, treatment increases the probability of a subject surviving for at least 2000 days after initiation of treatment. In an example, the increased probability is determined relative to a subject that is not treated with a composition of the disclosure. In an example, the increased probability is determined relative to a subject that has Class III heart failure.

In an example, treatment reduces the chance or risk of heart failure-related Major Adverse Cardiac Events (HF-MACE) defined as a composite of cardiac related death or resuscitated cardiac death, or non-fatal decompensated heart failure events. In an example, the chance or risk of HF-MACE is reduced over at least 6 months, at least 12 months, at least 24 months, at least 36 months after administration of a composition disclosed herein. In an example, treatment reduces the chance or risk of all-cause mortality.

### Ischemic Events

In an example, the present disclosure relates to a method of reducing the risk or incidence of ischemic events in subjects, in particular subjects with cardiomyopathy. In an example, the present disclosure relates to a method of reducing the risk or incidence of ischemic events in subjects with cardiomyopathy and elevated CRP. In an example, risk or incidence is reduced relative to a subject that does not receive a composition of the disclosure. For example, risk or incidence can be reduced relative to an untreated subject. In an example, the ischemic event is caused by the formation of an occlusion. In an example, the occlusion is an arterial occlusion. In an example, the ischemic event is formation of a cerebrovascular occlusion. In another example, the ischemic event is a formation of a cardiac occlusion. For example, the occlusion can form in the coronary artery.

Examples of ischemic events caused by formation of an occlusion include heart attack and stroke. Accordingly, in an example, the present disclosure relates to methods of reducing the risk or incidence of heart attack or stroke in a subject with cardiomyopathy.

The risk or incidence of ischemic events in subjects with cardiomyopathy is reduced by administering a cell therapy such as a composition of the disclosure.

In an example, the ischemic event is non-fatal. In an example, the ischemic event is fatal and, in this example, the method of the disclosure reduces risk of cardiac death from the ischemic event. Accordingly, in an example, the methods of the disclosure encompass a method of reducing risk of cardiac death or a non-fatal ischemic event in a subject, the method comprising administering to the subject a composition comprising mesenchymal lineage precursor or stem cells. In an example, the subject has one or more or all of:
class II heart failure
micro-vascular disease and/or macro-vascular disease;
active inflammation.

For example, the present disclosure encompasses a method of reducing risk of cardiac death or a non-fatal ischemic event in a subject, the method comprising administering to the subject a composition comprising mesenchymal lineage precursor or stem cells, wherein the subject has class II heart failure.

In another example, the present disclosure encompasses a method of reducing risk of cardiac death or a non-fatal ischemic event in a subject, the method comprising administering to the subject a composition comprising mesenchymal lineage precursor or stem cells, wherein the subject has micro-vascular disease and/or macro-vascular disease.

In another example, the present disclosure encompasses a method of reducing risk of cardiac death or a non-fatal ischemic event in a subject, the method comprising administering to the subject a composition comprising mesenchymal lineage precursor or stem cells, wherein the subject has active inflammation.

In an example, the subject has non-ischemic cardiomyopathy. For example, the subject's cardiomyopathy may be caused by an enlarged left ventricle (dilated cardiomyopathy. In another example, the cardiomyopathy is caused by a viral infection.

In another example, the subject has Class II or Class III heart failure according to the New York Heart Association (NYHA) classification scale.

In another example, the subject's level of N-terminal pro-B-type natriuretic peptide (NT-proBNP) is between 1000 pg/ml and 2000 pg/ml prior to administering the cells. In another example, the subject's C-reactive protein (CRP) level is elevated. In another example, the subject's CRP level is ≥1.5 mg/L. In another example, the subject's CRP level is ≥2 mg/L. In another example, the subject's CRP level is between 1 and 5 mg/L. In another example, the subject's CRP level is between 3 and 5 mg/L.

In an example, the cells are administered transendocardially.

In an example, reduced risk is reduced 3 year risk. In another example, the reduced risk is reduced 5 year risk. In these examples, the risk of ischemic event is reduced over a defined period of time.

### Mesenchymal Lineage Precursor Cells

As used herein, the term “mesenchymal lineage precursor or stem cell (MLPSC)” refers to undifferentiated multipotent cells that have the capacity to self-renew while maintaining multipotency and the capacity to differentiate into a number of cell types either of mesenchymal origin, for example, osteoblasts, chondrocytes, adipocytes, stromal cells, fibroblasts and tendons, or non-mesodermal origin, for example, hepatocytes, neural cells and epithelial cells. For the avoidance of doubt, a “mesenchymal lineage precursor cell” refers to a cell which can differentiate into a mesenchymal cell such as bone, cartilage, muscle and fat cells, and fibrous connective tissue.

The term “mesenchymal lineage precursor or stem cells” includes both parent cells and their undifferentiated progeny. The term also includes mesenchymal precursor cells, multipotent stromal cells, mesenchymal stem cells (MSCs), perivascular mesenchymal precursor cells, and their undifferentiated progeny.

Mesenchymal lineage precursor or stem cells can be autologous, allogeneic, xenogenic, syngeneic or isogenic. Autologous cells are isolated from the same individual to which they will be reimplanted. Allogeneic cells are isolated from a donor of the same species. Xenogenic cells are isolated from a donor of another species. Syngeneic or isogenic cells are isolated from genetically identical organisms, such as twins, clones, or highly inbred research animal models.

In an example, the mesenchymal lineage precursor or stem cells are allogeneic. In an example, the allogeneic mesenchymal lineage precursor or stem cells are culture expanded and cryopreserved.

Mesenchymal lineage precursor or stem cells reside primarily in the bone marrow, but have also shown to be present in diverse host tissues including, for example, cord blood and umbilical cord, adult peripheral blood, adipose tissue, trabecular bone and dental pulp. They are also found in skin, spleen, pancreas, brain, kidney, liver, heart, retina, brain, hair follicles, intestine, lung, lymph node, thymus, ligament, tendon, skeletal muscle, dermis, and periosteum; and are capable of differentiating into germ lines such as mesoderm and/or endoderm and/or ectoderm. Thus, mesenchymal lineage precursor or stem cells are capable of differentiating into a large number of cell types including, but not limited to, adipose, osseous, cartilaginous, elastic, muscular, and fibrous connective tissues. The specific lineage-commitment and differentiation pathway which these cells enter depends upon various influences from mechanical influences and/or endogenous bioactive factors, such as growth factors, cytokines, and/or local microenvironmental conditions established by host tissues.

The terms “enriched”, “enrichment” or variations thereof are used herein to describe a population of cells in which the proportion of one particular cell type or the proportion of a number of particular cell types is increased when compared with an untreated population of the cells (e.g., cells in their native environment). In one example, a population enriched for mesenchymal lineage precursor or stem cells comprises at least about 0.1% or 0.5% or 1% or 2% or 5% or 10% or 15% or 20% or 25% or 30% or 50% or 75% mesenchymal lineage precursor or stem cells. In this regard, the term “population of cells enriched for mesenchymal lineage precursor or stem cells” will be taken to provide explicit support for the term “population of cells comprising X % mesenchymal lineage precursor or stem cells”, wherein X % is a percentage as recited herein. The mesenchymal lineage precursor or stem cells can, in some examples, form clonogenic colonies, e.g. CFU-F (fibroblasts) or a subset thereof (e.g., 50% or 60% or 70% or 70% or 90% or 95%) can have this activity.

In an example of the present disclosure, the mesenchymal lineage precursor or stem cells are mesenchymal stem cells (MSCs). The MSCs may be a homogeneous composition or may be a mixed cell population enriched in MSCs. Homogeneous MSC compositions may be obtained by culturing adherent marrow or periosteal cells, and the MSCs may be identified by specific cell surface markers which are identified with unique monoclonal antibodies. A method for obtaining a cell population enriched in MSCs is described, for example, in U.S. Pat. No. 5,486,359. Alternative sources for MSCs include, but are not limited to, blood, skin, cord blood, muscle, fat, bone, and perichondrium. In an example, the MSCs are allogeneic. In an example, the MSCs are cryopreserved. In an example, the MSCs are culture expanded and cryopreserved.

In another example, the mesenchymal lineage precursor or stem cells are CD29+, CD54+, CD73+, CD90+, CD102+, CD105+, CD106+, CD166+, MHC1+MSCs.

Isolated or enriched mesenchymal lineage precursor or stem cells can be expanded in vitro by culture. Isolated or enriched mesenchymal lineage precursor or stem cells can be cryopreserved, thawed and subsequently expanded in vitro by culture.

In one example, isolated or enriched mesenchymal lineage precursor or stem cells are seeded at 50,000 viable cells/cm² in culture medium (serum free or serum-supplemented), for example, alpha minimum essential media (aMEM) supplemented with 5% fetal bovine serum (FBS) and glutamine, and allowed to adhere to the culture vessel overnight at 37° C., 20% O₂. The culture medium is subsequently replaced and/or altered as required and the cells cultured for a further 68 to 72 hours at 37° C., 5% O₂.

As will be appreciated by those of skill in the art, cultured mesenchymal lineage precursor or stem cells are phenotypically different to cells in vivo. For example, in one embodiment they express one or more of the following markers, CD44, NG2, DC146 and CD140b. Cultured mesenchymal lineage precursor or stem cells are also biologically different to cells in vivo, having a higher rate of proliferation compared to the largely non-cycling (quiescent) cells in vivo.

In one example, the population of cells is enriched from a cell preparation comprising STRO-1+ cells in a selectable form. In this regard, the term “selectable form” will be understood to mean that the cells express a marker (e.g., a cell surface marker) permitting selection of the STRO-1+ cells. The marker can be STRO-1, but need not be. For example, as described and/or exemplified herein, cells (e.g., mesenchymal precursor cells) expressing STRO-2 and/or STRO-3 (TNAP) and/or STRO-4 and/or VCAM-1 and/or CD146 and/or 3G5 also express STRO-1 (and can be STRO-1bright). Accordingly, an indication that cells are STRO-1+ does not mean that the cells are selected solely by STRO-1 expression. In one example, the cells are selected based on at least STRO-3 expression, e.g., they are STRO-3+ (TNAP+). For example, the MPCs can be isolated from bone mononuclear cells with an anti-STRO-3 antibody.

Reference to selection of a cell or population thereof does not necessarily require selection from a specific tissue source. As described herein STRO-1+ cells can be selected from or isolated from or enriched from a large variety of sources. That said, in some examples, these terms provide support for selection from any tissue comprising STRO-1+ cells (e.g., mesenchymal precursor cells) or vascularized tissue or tissue comprising pericytes (e.g., STRO-1+ pericytes) or any one or more of the tissues recited herein.

In one example, the cells used in the present disclosure express one or more markers individually or collectively selected from the group consisting of TNAP+, VCAM-1+, THY-1+, STRO-2+, STRO-4+ (HSP-90B), CD45+, CD146+, 3G5+ or any combination thereof.

By “individually” is meant that the disclosure encompasses the recited markers or groups of markers separately, and that, notwithstanding that individual markers or groups of markers may not be separately listed herein the accompanying claims may define such marker or groups of markers separately and divisibly from each other.

By “collectively” is meant that the disclosure encompasses any number or combination of the recited markers or groups of markers, and that, notwithstanding that such numbers or combinations of markers or groups of markers may not be specifically listed herein the accompanying claims may define such combinations or sub-combinations separately and divisibly from any other combination of markers or groups of markers.

As used herein the term “TNAP” is intended to encompass all isoforms of tissue non-specific alkaline phosphatase. For example, the term encompasses the liver isoform (LAP), the bone isoform (BAP) and the kidney isoform (KAP). In one example, the TNAP is BAP. In one example, TNAP as used herein refers to a molecule which can bind the STRO-3 antibody produced by the hybridoma cell line deposited with ATCC on 19 Dec. 2005 under the provisions of the Budapest Treaty under deposit accession number PTA-7282.

Furthermore, in one example, the STRO-1+ cells are capable of giving rise to clonogenic CFU-F.

In one example, a significant proportion of the STRO-1+ cells are capable of differentiation into at least two different germ lines. Non-limiting examples of the lineages to which the STRO-1+ cells may be committed include bone precursor cells; hepatocyte progenitors, which are multipotent for bile duct epithelial cells and hepatocytes; neural restricted cells, which can generate glial cell precursors that progress to oligodendrocytes and astrocytes; neuronal precursors that progress to neurons; precursors for cardiac muscle and cardiomyocytes, glucose-responsive insulin secreting pancreatic beta cell lines. Other lineages include, but are not limited to, odontoblasts, dentin-producing cells and chondrocytes, and precursor cells of the following: retinal pigment epithelial cells, fibroblasts, skin cells such as keratinocytes, dendritic cells, hair follicle cells, renal duct epithelial cells, smooth and skeletal muscle cells, testicular progenitors, vascular endothelial cells, tendon, ligament, cartilage, adipocyte, fibroblast, marrow stroma, cardiac muscle, smooth muscle, skeletal muscle, pericyte, vascular, epithelial, glial, neuronal, astrocyte and oligodendrocyte cells.

In an example, mesenchymal lineage precursor or stem cells are obtained from a single donor, or multiple donors where the donor samples or mesenchymal lineage precursor or stem cells are subsequently pooled and then culture expanded.

Mesenchymal lineage precursor or stem cells encompassed by the present disclosure may also be cryopreserved prior to administration to a subject. In an example, mesenchymal lineage precursor or stem cells are culture expanded and cryopreserved prior to administration to a subject.

In an example, the present disclosure encompasses mesenchymal lineage precursor or stem cells as well as progeny thereof, soluble factors derived therefrom, and/or extracellular vesicles isolated therefrom. In another example, the present disclosure encompasses mesenchymal lineage precursor or stem cells as well as extracellular vesicles isolated therefrom. For example, it is possible to culture expand mesenchymal precursor lineage or stem cells of the disclosure for a period of time and under conditions suitable for secretion of extracellular vesicles into the cell culture medium. Secreted extracellular vesicles can subsequently be obtained from the culture medium for use in therapy.

The term “extracellular vesicles” as used herein, refers to lipid particles naturally released from cells and ranging in size from about 30 nm to as a large as 10 microns, although typically they are less than 200 nm in size. They can contain proteins, nucleic acids, lipids, metabolites, or organelles from the releasing cells (e.g., mesenchymal stem cells; STRO-1⁺ cells).

The term “exosomes” as used herein, refers to a type of extracellular vesicle generally ranging in size from about 30 nm to about 150 nm and originating in the endosomal compartment of mammalian cells from which they are trafficked to the cell membrane and released. They may contain nucleic acids (e.g., RNA; microRNAs), proteins, lipids, and metabolites and function in intercellular communication by being secreted from one cell and taken up by other cells to deliver their cargo.

In an example, compositions of the disclosure comprise cells that induce new blood vessel formation in target tissue. In an example, the target tissue is the heart. In another example, the cells secrete factors that protect at risk or damaged myocardium. In an example, at risk or damaged myocardium has been subject to a lack of blood flow resulting from an ischemic event. In an example, the cells secrete factors that reduce apoptosis in cardiomyocytes.

### Culture Expansion of the Cells

In an example, mesenchymal lineage precursor or stem cells are culture expanded. “Culture expanded” mesenchymal lineage precursor or stem cells media are distinguished from freshly isolated cells in that they have been cultured in cell culture medium and passaged (i.e. sub-cultured). In an example, culture expanded mesenchymal lineage precursor or stem cells are culture expanded for about 4-10 passages. In an example, mesenchymal lineage precursor or stem cells are culture expanded for at least 5, at least 6, at least 7, at least 8, at least 9, at least 10 passages. For example, mesenchymal lineage precursor or stem cells can be culture expanded for at least 5 passages. In an example, mesenchymal lineage precursor or stem cells can be culture expanded for at least 5-10 passages. In an example, mesenchymal lineage precursor or stem cells can be culture expanded for at least 5-8 passages. In an example, mesenchymal lineage precursor or stem cells can be culture expanded for at least 5-7 passages. In an example, mesenchymal lineage precursor or stem cells can be culture expanded for more than 10 passages. In another example, mesenchymal lineage precursor or stem cells can be culture expanded for more than 7 passages. In these examples, stem cells may be culture expanded before being cryopreserved to provide an intermediate cryopreserved MLPSC population. In an example, compositions of the present disclosure are produced by culturing cells from an intermediate cryopreserved MLPSC population or, put another way, a cryopreserved intermediate.

In an example, compositions of the disclosure comprise mesenchymal lineage precursor or stem cells that are culture expanded from a cryopreserved intermediate. In an example, the cells culture expanded from a cryopreserved intermediate are culture expanded for at least 5, at least 6, at least 7, at least 8, at least 9, at least 10 passages. For example, mesenchymal lineage precursor or stem cells can be culture expanded for at least 5 passages. In an example, mesenchymal lineage precursor or stem cells can be culture expanded for at least 5-10 passages. In an example, mesenchymal lineage precursor or stem cells can be culture expanded for at least 5-8 passages. In an example, mesenchymal lineage precursor or stem cells can be culture expanded for at least 5-7 passages. In an example, mesenchymal lineage precursor or stem cells can be culture expanded for more than 10 passages. In another example, mesenchymal lineage precursor or stem cells can be culture expanded for more than 7 passages.

In an example, mesenchymal lineage precursor or stem cells culture expanded from a cryopreserved intermediate can be culture expanded in medium free of animal proteins. In an example, mesenchymal lineage precursor or stem cells culture expanded from a cryopreserved intermediate can be culture expanded in xeno-free medium. In an example, mesenchymal lineage precursor or stem cells culture expanded from a cryopreserved intermediate can be culture expanded in medium that is fetal bovine serum free.

In an embodiment, mesenchymal lineage precursor or stem cells can be obtained from a single donor, or multiple donors where the donor samples or mesenchymal lineage precursor or stem cells are subsequently pooled and then culture expanded. In an example, the culture expansion process comprises:
i. expanding by passage expansion the number of viable cells to provide a preparation of at least about 1 billion of the viable cells, wherein the passage expansion comprises establishing a primary culture of isolated mesenchymal lineage precursor or stem cells and then serially establishing a first non-primary (P1) culture of isolated mesenchymal lineage precursor or stem cells from the previous culture;
ii. expanding by passage expansion the P1 culture of isolated mesenchymal lineage precursor or stem cells to a second non-primary (P2) culture of mesenchymal lineage precursor or stem cells; and,
iii. preparing and cryopreserving an in-process intermediate mesenchymal lineage precursor or stem cells preparation obtained from the P2 culture of mesenchymal lineage precursor or stem cells; and,
iv. thawing the cryopreserved in-process intermediate mesenchymal lineage precursor or stem cells preparation and expanding by passage expansion the in-process intermediate mesenchymal lineage precursor or stem cells preparation.

In an example, the expanded mesenchymal lineage precursor or stem cell preparation has an antigen profile and an activity profile comprising:
i. less than about 0.75% CD45+ cells;
ii. at least about 95% CD105+ cells;
iii. at least about 95% CD166+ cells.

In an example, the expanded mesenchymal lineage precursor or stem cell preparation is capable of inhibiting IL2-Rα expression by CD3/CD28-activated PBMCs by at least about 30% relative to a control.

In an example, culture expanded mesenchymal lineage precursor or stem cells are culture expanded for about 4-10 passages, wherein the mesenchymal lineage precursor or stem cells have been cryopreserved after at least 2 or 3 passages before being further culture expanded. In an example, mesenchymal lineage precursor or stem cells are culture expanded for at least 1, at least 2, at least 3, at least 4, at least 5 passages, cryopreserved and then further culture expanded for at least 1, at least 2, at least 3, at least 4, at least 5 passages before being cultured according to the methods of the disclosure.

The process of mesenchymal lineage precursor or stem cell isolation and ex vivo expansion can be performed using any equipment and cell handing methods known in the art. Various culture expansion embodiments of the present disclosure employ steps that require manipulation of cells, for example, steps of seeding, feeding, dissociating an adherent culture, or washing. Any step of manipulating cells has the potential to insult the cells. Although mesenchymal lineage precursor or stem cells can generally withstand a certain amount of insult during preparation, cells are preferably manipulated by handling procedures and/or equipment that adequately performs the given step(s) while minimizing insult to the cells.

In an example, mesenchymal lineage precursor or stem cells are washed in an apparatus that includes a cell source bag, a wash solution bag, a recirculation wash bag, a spinning membrane filter having inlet and outlet ports, a filtrate bag, a mixing zone, an end product bag for the washed cells, and appropriate tubing, for example, as described in U.S. Pat. No. 6,251,295, which is hereby incorporated by reference.

In an example, a mesenchymal lineage precursor or stem cell composition cultured according to the present disclosure is 95% homogeneous with respect to being CD105 positive and CD166 positive and being CD45 negative. In an example, this homogeneity persists through ex vivo expansion; i.e. though multiple population doublings.

In an example, mesenchymal lineage precursor or stem cells of the disclosure are culture expanded in 3D culture. For example, mesenchymal lineage precursor or stem cells of the disclosure can be culture expanded in a bioreactor. In an example, mesenchymal lineage precursor or stem cells of the disclosure are initially culture expanded in 2D culture prior to being further expanded in 3D culture. In an example, mesenchymal lineage precursor or stem cells of the disclosure are culture expanded from a master cell bank. In an example, mesenchymal lineage precursor or stem cells of the disclosure are culture expanded from a master cell bank in 2D culture before seeding in 3D culture. In an example, mesenchymal lineage precursor or stem cells of the disclosure are culture expanded from a master cell bank in 2D culture for at least 3 days before seeding in 3D culture in a bioreactor. In an example, mesenchymal lineage precursor or stem cells of the disclosure are culture expanded from a master cell bank in 2D culture for at least 4 days before seeding in 3D culture in a bioreactor. In an example, mesenchymal lineage precursor or stem cells of the disclosure are culture expanded from a master cell bank in 2D culture for between 3 and 5 days before seeding in 3D culture in a bioreactor. In these examples, 2D culture can be performed in a cell factory. Various cell factory products are available commercially (e.g. Thermofisher, Sigma).

In an example, cells of the disclosure are STRO-3+ before they are culture expanded to provide a cryopreserved intermediate. In this example, the intermediate can be subsequently defrosted and subject to further culture expansion (e.g. culture expansion to drug product).

### Cell Culture Medium

Mesenchymal lineage precursor or stem cells disclosed herein can be culture expanded in various suitable growth mediums.

The term “medium” or “media” as used in the context of the present disclosure, includes the components of the environment surrounding the cells. The media contributes to and/or provides the conditions suitable to allow cells to grow. Media may be solid, liquid, gaseous or a mixture of phases and materials. Media can include liquid growth media as well as liquid media that do not sustain cell growth. Media also include gelatinous media such as agar, agarose, gelatin and collagen matrices. Exemplary gaseous media include the gaseous phase that cells growing on a petri dish or other solid or semisolid support are exposed to.

The cell culture media used for culture expansion contains all essential amino acids and may also contain non-essential amino acids. In general, amino acids are classified into essential amino acids (Thr, Met, Val, Leu, Ile, Phe, Trp, Lys, His) and non-essential amino acids (Gly, Ala, Ser, Cys, Gln, Asn, Asp, Tyr, Arg, Pro).

Those of skill in the art will appreciate that for optimal results, the basal medium must be appropriate for the cell line of interest. For example, it may be necessary to increase the level of glucose (or other energy source) in the basal medium, or to add glucose (or other energy source) during the course of culture, if this energy source is found to be depleted and to thus limit growth. In an example, dissolved oxygen (DO) levels can also be controlled.

In an example, the cell culture medium contains human derived additives. For example, human serum and human platelet cell lysate can be added to the cell culture media.

In an example, the cell culture medium contains only human derived additives. Thus, in an example, the cell culture media is xeno-free. For avoidance of doubt, in these examples, the culture medium is free of animal proteins. In an example, cell culture medium used in the methods of the disclosure is free of animal components.

In an example, the culture medium comprises serum. In other examples the culture medium is fetal bovine serum free culture medium comprising growth factors that promote mesenchymal lineage precursor or stem cell proliferation. In an embodiment, the culture medium is serum free stem cell culture medium. In an example, the cell culture medium comprises:
a basal medium;
platelet derived growth factor (PDGF);
fibroblast growth factor 2 (FGF2).

In an example, the culture medium comprises platelet derived growth factor (PDGF) and fibroblast growth factor 2 (FGF2), wherein the level of FGF2 is less than about 6 ng/ml. For example, the FGF2 level may be less than about 5 ng/ml, less than about 4 ng/ml, less than about 3 ng/ml, less than about 2 ng/ml, less than about 1 ng/ml. In other examples, the FGF2 level is less than about 0.9 ng/ml, less than about 0.8 ng/ml, less than about 0.7 ng/ml, less than about 0.6 ng/ml, less than about 0.5 ng/ml, less than about 0.4 ng/ml, less than about 0.3 ng/ml, less than about 0.2 ng/ml.

In another example, the level of FGF2 is between about 1 pg/ml and 100 pg/ml. In another example, the level of FGF2 is between about 5 pg/ml and 80 pg/ml.

In an example, the PDGF is PDGF-BB. In an example, the level of PDGF-BB is between about 1 ng/ml and 150 ng/ml. In another example, the level of PDGF-BB is between about 7.5 ng/ml and 120 ng/ml. In another example, the level of PDGF-BB is between about 15 ng/ml and 60 ng/ml. In another example, the level of PDGF-BB is at least about 10 ng/ml. In another example, the level of PDGF-BB is at least about 15 ng/ml. In another example, the level of PDGF-BB is at least about 20 ng/ml. In another example, the level of PDGF-BB is at least about 21 ng/ml. In another example, the level of PDGF-BB is at least about 22 ng/ml. In another example, the level of PDGF-BB is at least about 23 ng/ml. In another example, the level of PDGF-BB is at least about 24 ng/ml. In another example, the level of PDGF-BB is at least about 25 ng/ml.

In another example, the PDGF is PDGF-AB. In an example, the level of PDGF-AB is between about 1 ng/ml and 150 ng/ml. In another example, the level of PDGF-AB is between about 7.5 ng/ml and 120 ng/ml. In another example, the level of PDGF-AB is between about 15 ng/ml and 60 ng/ml. In another example, the level of PDGF-AB is at least about 10 ng/ml. In another example, the level of PDGF-AB is at least about 15 ng/ml. In another example, the level of PDGF-AB is at least about 20 ng/ml. In another example, the level of PDGF-AB is at least about 21 ng/ml. In another example, the level of PDGF-AB is at least about 22 ng/ml. In another example, the level of PDGF-AB is at least about 23 ng/ml. In another example, the level of PDGF-AB is at least about 24 ng/ml. In another example, the level of PDGF-AB is at least about 25 ng/ml.

In other examples, additional factors can be added to the cell culture medium. In an example, the culture medium further comprising EGF. EGF is a growth factor that stimulates cell proliferation by binding to its receptor EGFR. In an example, the method of the present disclosure comprises culturing a population of stem cells in a fetal bovine serum free cell culture medium further comprising EGF. In an example, the level of EGF is between about 0.1 and 7 ng/ml. For example, the level of EGF can be at least about 5 ng/ml.

In another example, the level of EGF is between about 0.2 ng/ml and 3.2 ng/ml. In another example, the level of EGF is between about 0.4 ng/ml and 1.6 ng/ml. In another example, the level of EGF is between about 0.2 ng/ml. In another example, the level of EGF is at least about 0.3 ng/ml. In another example, the level of EGF is at least about 0.4 ng/ml. In another example, the level of EGF is at least about 0.5 ng/ml. In another example, the level of EGF is at least about 0.6 ng/ml. In another example, the level of EGF is at least about 0.7 ng/ml. In another example, the level of EGF is at least about 0.8 ng/ml. In another example, the level of EGF is at least about 0.9 ng/ml. In another example, the level of EGF is at least about 1.0 ng/ml.

In the above examples, basal medium such as Alpha MEM or StemSpan™ can be supplemented with the referenced quantity of growth factor. In an example, the culture medium comprises Alpha MEM or StemSpan™ supplemented with 32 ng/ml PDGF-BB, 0.8 ng/ml EGF and 0.02 ng/ml FGF.

In other examples, additional factors can be added to the cell culture medium. For example, the cell culture media can be supplemented with one or more stimulatory factors selected from the group consisting of epidermal growth factor (EGF), 1α,25-dihydroxyvitamin D3 (1,25D), tumor necrosis factor α (TNF-α), interleukin-1β (IL-1β) and stromal derived factor 1α (SDF-1α). In another embodiment, cells may also be cultured in the presence of at least one cytokine in an amount adequate to support growth of the cells. In another embodiment, cells can be cultured in the presence of heparin or a derivative thereof. For example, the cell culture medium may contain about 50 ng/ml of heparin. In other examples, the cell culture medium contains about 60 ng/ml of heparin, about 70 ng/ml of heparin, about 80 ng/ml of heparin, about 90 ng/ml of heparin, about 100 ng/ml of heparin, about 110 ng/ml of heparin, about 110 ng/ml of heparin, about 120 ng/ml of heparin, about 130 ng/ml of heparin, about 140 ng/ml of heparin, about 150 ng/ml of heparin or a derivative thereof. In an example, the heparin derivative is a sulphate). Various forms of heparin sulphate are known in the art and include heparin sulphate 2 (HS2). HS2 can be derived from various sources including for example, the liver of male and/or female mammals. Thus, an exemplary heparin sulphate includes male liver heparin sulphate (MML HS) and female liver heparin sulphate (FML HS).

In another example, the cell culture medium of the present disclosure promotes stem cell proliferation while maintaining stem cells in an undifferentiated state. Stem cells are considered to be undifferentiated when they have not committed to a specific differentiation lineage. As discussed above, stem cells display morphological characteristics that distinguish them from differentiated cells. Furthermore, undifferentiated stem cells express genes that may be used as markers to detect differentiation status. The polypeptide products may also be used as markers to detect differentiation status. Accordingly, one of skill in the art could readily determine whether the methods of the present disclosure maintain stem cells in an undifferentiated state using routine morphological, genetic and/or proteomic analysis.

### Modification of the Cells

The mesenchymal lineage precursor or stem cells disclosed herein may be altered in such a way that upon administration, lysis of the cell is inhibited. Alteration of an antigen can induce immunological non-responsiveness or tolerance, thereby preventing the induction of the effector phases of an immune response (e.g., cytotoxic T cell generation, antibody production etc.) which are ultimately responsible for rejection of foreign cells in a normal immune response. Antigens that can be altered to achieve this goal include, for example, MHC class I antigens, MHC class II antigens, LFA-3 and ICAM-1.

The mesenchymal lineage precursor or stem cells may also be genetically modified to express proteins of importance for the differentiation and/or maintenance of striated skeletal muscle cells. Exemplary proteins include growth factors (TGF-β, insulin-like growth factor 1 (IGF-1), FGF), myogenic factors (e.g. myoD, myogenin, myogenic factor 5 (Myf5), myogenic regulatory factor (MRF)), transcription factors (e.g. GATA-4), cytokines (e.g. cardiotropin-1), members of the neuregulin family (e.g. neuregulin 1, 2 and 3) and homeobox genes (e.g. Csx, tinman and NKx family).

### Compositions

Mesenchymal lineage or stem cells disclosed herein can be culture expanded from a cryopreserved intermediate to produce a preparation containing at least one therapeutic dose.

In an example, compositions of the disclosure comprise between 10×10⁶ cells and 35×10⁶ cells. In another example, the composition comprises between 20×10⁶ cells and 30×10⁶ cells. In other examples, the composition comprises at least 100×10⁶ cells. In another example, the composition comprises between 50×10⁶ cells and 500×10⁶ cells. In other examples, compositions of the disclosure comprise 150 million cells.

In one example, compositions of the disclose comprise a pharmaceutically acceptable carrier and/or excipient. The terms “carrier” and “excipient” refer to compositions of matter that are conventionally used in the art to facilitate the storage, administration, and/or the biological activity of an active compound (see, e.g., Remington's Pharmaceutical Sciences, 16th Ed., Mac Publishing Company (1980). A carrier may also reduce any undesirable side effects of the active compound. A suitable carrier is, for example, stable, e.g., incapable of reacting with other ingredients in the carrier. In one example, the carrier does not produce significant local or systemic adverse effect in recipients at the dosages and concentrations employed for treatment.

Suitable carriers for the present disclosure include those conventionally used, e.g., water, saline, aqueous dextrose, lactose, Ringer's solution, a buffered solution, hyaluronan and glycols are exemplary liquid carriers, particularly (when isotonic) for solutions. Suitable pharmaceutical carriers and excipients include starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like.

In another example, a carrier is a media composition, e.g., in which a cell is grown or suspended. Such a media composition does not induce any adverse effects in a subject to whom it is administered. Exemplary carriers and excipients do not adversely affect the viability of a cell and/or the ability of a cell to treat or prevent disease.

In one example, the carrier or excipient provides a buffering activity to maintain the cells and/or soluble factors at a suitable pH to thereby exert a biological activity, e.g., the carrier or excipient is phosphate buffered saline (PBS). PBS represents an attractive carrier or excipient because it interacts with cells and factors minimally and permits rapid release of the cells and factors, in such a case, the composition of the disclosure may be produced as a liquid for direct application to the blood stream or into a tissue or a region surrounding or adjacent to a tissue, e.g., by injection.

Compositions of the disclosure may be cryopreserved. Cryopreservation of mesenchymal lineage precursor or stem cells can be carried out using slow-rate cooling methods or ‘fast’ freezing protocols known in the art. Preferably, the method of cryopreservation maintains similar phenotypes, cell surface markers and growth rates of cryopreserved cells in comparison with unfrozen cells.

The cryopreserved composition may comprise a cryopreservation solution. The pH of the cryopreservation solution is typically 6.5 to 8, preferably 7.4.

The cryopreservation solution may comprise a sterile, non-pyrogenic isotonic solution such as, for example, PlasmaLyte ATM. 100 mL of PlasmaLyte ATM contains 526 mg of sodium chloride, USP (NaCl); 502 mg of sodium gluconate (C6H11NaO7); 368 mg of sodium acetate trihydrate, USP (C2H3NaO2·3H2O); 37 mg of potassium chloride, USP (KCl); and 30 mg of magnesium chloride, USP (MgCl2·6H2O). It contains no antimicrobial agents. The pH is adjusted with sodium hydroxide. The pH is 7.4 (6.5 to 8.0).

The cryopreservation solution may comprise Profreeze™. The cryopreservation solution may additionally or alternatively comprise culture medium, for example, aMEM.

To facilitate freezing, a cryoprotectant such as, for example, dimethylsulfoxide (DMSO), is usually added to the cryopreservation solution. Ideally, the cryoprotectant should be nontoxic for cells and patients, nonantigenic, chemically inert, provide high survival rate after thawing and allow transplantation without washing. However, the most commonly used cryoprotector, DMSO, shows some cytotoxicity. Hydroxylethyl starch (HES) may be used as a substitute or in combination with DMSO to reduce cytotoxicity of the cryopreservation solution.

The cryopreservation solution may comprise one or more of DMSO, hydroxyethyl starch, human serum components and other protein bulking agents. In one example, the cryopreserved solution comprises Plasma-Lyte A (70%), DMSO (10%), HSA (25%) solution, the HSA solution comprising 5% HSA and 15% buffer.

In an example, the cryopreservation solution may further comprise one or more of methylcellulose, polyvinyl pyrrolidone (PVP) and trehalose.

The cryopreserved composition may be thawed and administered directly to the subject or added to another solution, for example, comprising hyaluronic acid. Alternatively, the cryopreserved composition may be thawed and the mesenchymal lineage precursor or stem cells resuspended in an alternate carrier prior to administration.

The compositions described herein may be administered alone or as admixtures with other cells. The cells of different types may be admixed with a composition of the disclosure immediately or shortly prior to administration, or they may be co-cultured together for a period of time prior to administration.

In one example, the composition comprises an effective amount or a therapeutically or prophylactically effective amount of mesenchymal lineage precursor or stem cells and/or progeny thereof and/or soluble factor derived therefrom. For example, the composition comprises about 1×10⁵ stem cells to about 1×10⁹ stem cells or about 1.25×103 stem cells to about 1.25×10⁷ stem cells/kg (80 kg subject). The exact amount of cells to be administered is dependent upon a variety of factors, including the age, weight, and sex of the subject, and the extent and severity of the disorder being treated.

Despite the number of cells provided in the composition, in an example, 50×10⁶ to 200×10⁷ cells are administered. In other examples, 60×10⁶ to 200×10⁶ cells or 75×10⁶ to 150×10⁶ cells are administered. In an example, 75×10⁶ cells are administered. In another example, 150×10⁶ cells are administered.

In an example, the composition comprises greater than 5.00×10⁶ viable cells/mL. In another example, the composition comprises greater than 5.50×10⁶ viable cells/mL. In another example, the composition comprises greater than 6.00×10⁶ viable cells/mL. In another example, the composition comprises greater than 6.50×10⁶ viable cells/mL. In another example, the composition comprises greater than 6.68×10⁶ viable cells/mL.

In an example, the mesenchymal lineage precursor or stem cells comprise at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% of the cell population of the composition.

In an example, the composition may optionally be packaged in a suitable container with written instructions for a desired purpose.

Compositions of the disclosure may be administered systemically, such as, for example, by intravenous administration. In an example, compositions are administered transendocardially.

### Risk of Cardiac Death, Myocardial Infarction or Stroke

In an example, the methods of the present disclosure relate to methods of assessing risk of one or more of cardiac death, myocardial infarction or stroke based on the level of CRP in a subject. For example, the methods of the present disclosure can relate to methods of assessing risk of cardiac death based on the level of CRP in a subject. In an example, the present disclosure encompasses a method for determining elevated risk of one or more of cardiac death, myocardial infarction or stroke in a subject, the method comprising measuring the level of CRP in a sample obtained from a subject, wherein elevated CRP indicates elevated risk of cardiac death, myocardial infarction or stroke. In an example, the subject has progressive heart failure. For example, the subject can have NYHA Class II progressive heart failure. Accordingly, in an example, the sample is obtained from a subject with NYHA Class II progressive heart failure. In an example, the sample is a blood sample.

In an example, a level of CRP >1 mg/L indicates elevated risk of cardiac death, myocardial infarction or stroke. In an example, a level of CRP >1.5 mg/L indicates elevated risk of cardiac death, myocardial infarction or stroke. In an example, a level of CRP ≥2 mg/L indicates elevated risk of cardiac death, myocardial infarction or stroke. In an example, a level of CRP between ≥2 and 5 mg/L indicates elevated risk of cardiac death, myocardial infarction or stroke. In an example, the level of CRP is measured after an ischemic event. In an example, the ischemic event is a myocardial infarction.

In an example, a composition comprising cells which induce new blood vessel formation in target tissue is administered to subjects assessed as having elevated risk of cardiac death, myocardial infarction or stroke. Accordingly, in an example, the present disclosure relates to a method of treating progressive heart failure, the method comprising i) selecting a subject having Class II heart failure according to the New York Heart Association (NYHA) classification scale and elevated CRP level, and ii) administering to the subject a composition according to the present disclosure. In an example, the subject's CRP level is ≥2 mg/ml.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

The present application claims priority from AU2021903706 filed 17 Nov. 2021 and U.S. 63/384,200 filed on 17 Nov. 2022, the disclosures of which are incorporated herein by reference.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### Examples

### Composition

The composition consists of human bone marrow-derived allogeneic MPCs isolated from bone mononuclear cells with anti-STRO-3 antibodies, expanded ex vivo, and cryopreserved.

### Patients

### Baseline Data

All patients (18-80 years old) had heart failure with reduced ejection fraction (HFrEF), a progressive disease that leads to recurrent hospitalizations and increased risk of death.

| | | Total | | | | | |
|---|---|---|---|---|---|---|---|
| Parameter | Units | (n = 537) | | NYHA II | | NYHA III | |
| NYHA Functional Class | Number, % | | | 206 | 38.4% | 331 | 61.6% |
| II | Number, % | 206 | 38.4% | | | | |
| III | Number, % | 331 | 61.6% | | | | |
| Age (537 unique subjects) | Yrs (mean) | 62.7 | 64.0 | 62.2 | 63.0 | 63.1 | 65.0 |
| Sex (537 unique subjects)* | | | | | | | |
| Males | Number, % | 428 | 79.7% | 168 | 81.6% | 260 | 78.5% |
| Females | Number, % | 109 | 20.3% | 38 | 18.4% | 71 | 21.5% |
| Race (537 unique subjects) | | | | | | | |
| White | Number, % | 414 | 77.1% | 166 | 80.6% | 248 | 74.9% |
| Black | Number, % | 99 | 18.4% | 34 | 16.5% | 65 | 19.6% |
| Asian | Number, % | 7 | 1.3% | 2 | 1.0% | 5 | 1.5% |
| Other | Number, % | 17 | 3.2% | 4 | 1.9% | 13 | 3.9% |
| Ethnic Group (537 unique subjects) | | | | | | | |
| Hispanic | Number, % | 34 | 6.3% | 11 | 5.3% | 23 | 6.9% |
| Blood pressure | | | | | | | |
| Systolic | mmHg (mean) | 117.1 | 114.0 | 117.2 | 116 | 117 | 113 |
| Diastolic | mmHg (mean) | 70.2 | 70.0 | 71.2 | 71 | 69.5 | 68 |
| Heart rate | Beats/min (mean) | 72.3 | 71.0 | 70.9 | 70 | 73.1 | 71 |
| Body weight | kg (mean, median) | 92.8 | 90.9 | 91.8 | 89.2 | 93.4 | 92.5 |
| Non-Ischemic vs Ischemic Cardiomyopathy (per baseline CRF) | | | | | | | |
| Ischemic | Number, % | 303 | 56.4% | 111 | 53.9% | 192 | 58.0% |
| Non-ischemic | Number, % | 234 | 43.6% | 95 | 46.1% | 139 | 42.0% |
| Past Myocardial infarction | Number, % | 280 | 52.1% | 106 | 51.5% | 174 | 52.6% |
| Coronary revascularization | | | | | | | |
| Previous PCI and/or | Number, % | 307 | 57.2% | 119 | 57.8% | 188 | 56.8% |
| CABG (unique subjects) | | | | | | | |
| Echocardiographic Imaging (530 unique subjects) | | | | | | | |
| Left ventricular ejection | % (mean, median) | 28.6 | 28.6 | 28.6 | 29.0 | 28.6 | 28.5 |
| fraction | | | | | | | |
| Left ventricular end- | mL (mean, median) | 149.8 | 135.9 | 155.4 | 137.4 | 146.2 | 135.0 |
| systolic volume | | | | | | | |
| <=100 | number, % | 101 | 18.8% | 41 | 19.9% | 60 | 18.1% |
| >100 | number, % | 429 | 79.9% | 163 | 79.1% | 266 | 80.4% |
| missing | number, % | 7 | 1.3% | 2 | 1.0% | 5 | 1.5% |
| Left ventricular end- | mL (mean, median) | 206.5 | 194.0 | 213.1 | 193.7 | 202.3 | 194.3 |
| diastolic volume | | | | | | | |
| 6MWT distance (537 | m (mean, median) | 331.5 | 340.5 | 356.8 | 367 | 311.7 | 329.2 |
| unique subjects) | | | | | | | |
| Biomarkers | | | | | | | |
| NT-proBNP | pg/mL (mean, median) | 2166 | 1400 | 1809 | 1322 | 2390 | 1458 |
| hsCRP | mg/L (mean, median) | 5.3 | 2.5 | 3.6 | 2 | 6.4 | 3.3 |
| AICD without CRT | Number, % | 245 | 84.0% | 99 | 81.6% | 146 | 85.5% |
| CRT-D | Number, % | 206 | | 69 | | 137 | |
| Laboratory measurements | | | | | | | |
| Sodium | mequiv/L (mean) | 139.5 | 140.0 | 139.5 | 140.0 | 139.6 | 140.0 |
| Potassium | mequiv/L (mean) | 4.5 | 4.5 | 4.5 | 4.5 | 4.5 | 4.5 |
| Creatinine | mg/dL (mean) | 1.2 | 1.1 | 1.1 | 1.1 | 1.3 | 1.2 |
| BUN | mg/dL (mean) | 24.2 | 21.0 | 22.7 | 20.5 | 25.2 | 22.0 |
| HbA1c | % (mean) | 6.4 | 6.1 | 6.3 | 6.0 | 6.4 | 6.2 |
| Hemoglobin | g/dL (mean) | 13.6 | 13.7 | 13.8 | 13.9 | 13.5 | 13.5 |
| Hematrocit | % (mean) | 41.8 | 41.8 | 42.3 | 42.4 | 41.5 | 41.1 |

Key inclusion criteria included a left ventricular ejection fraction ≤40% by two-dimensional echocardiogram or ≤35% by multi-gated acquisition scan. Furthermore, the study population was enriched at enrolment to target patients with more severe disease by the presence of one or more of the following: (a) at least 1 HF hospitalization between 1 and 9 months before screening, and/or (b) at least 1 outpatient urgent care HF visit requiring intravenous diuretic, vasodilator, and/or positive inotropic therapy between 1 and 9 months before screening, and/or (c) plasma levels of NT-proBNP >1000 pg/mL or >1200 pg/mL for patients with atrial fibrillation.

Primary and secondary outcome measurements were as follows:

### 1) Non-Fatal Major Adverse Cardiac Events (MACE):

Heart Failure MACE (recurrent decompensated heart failure events or high-grade arrhythmias);
Ischemic MACE (heart attacks or strokes).
   2) Death from Cardiac Causes

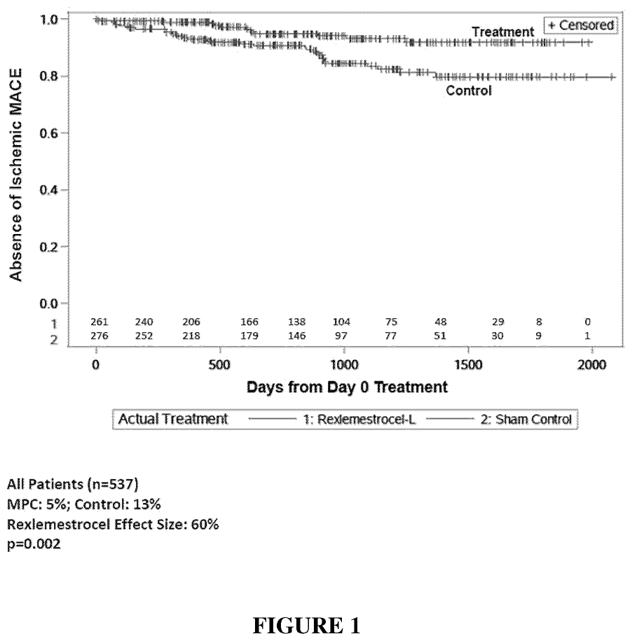

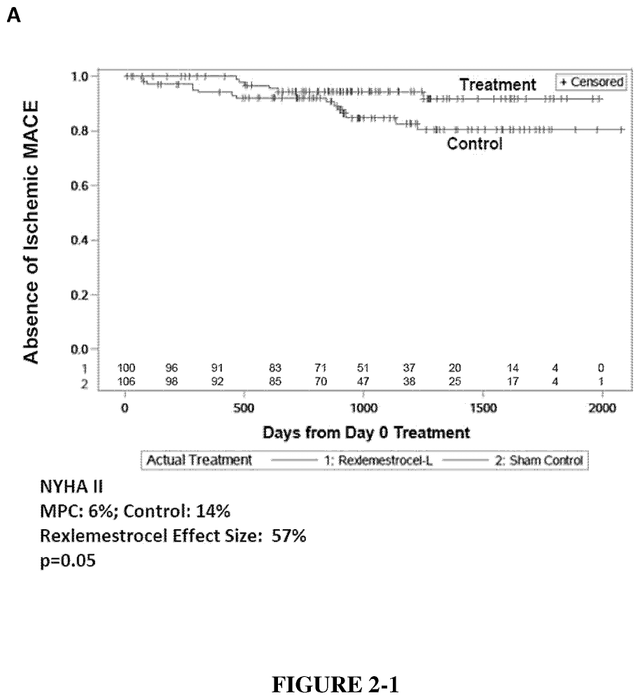

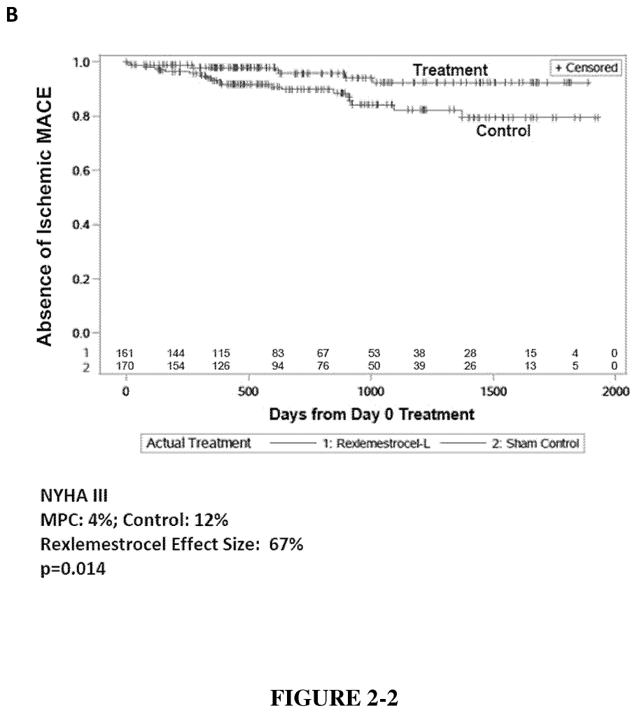

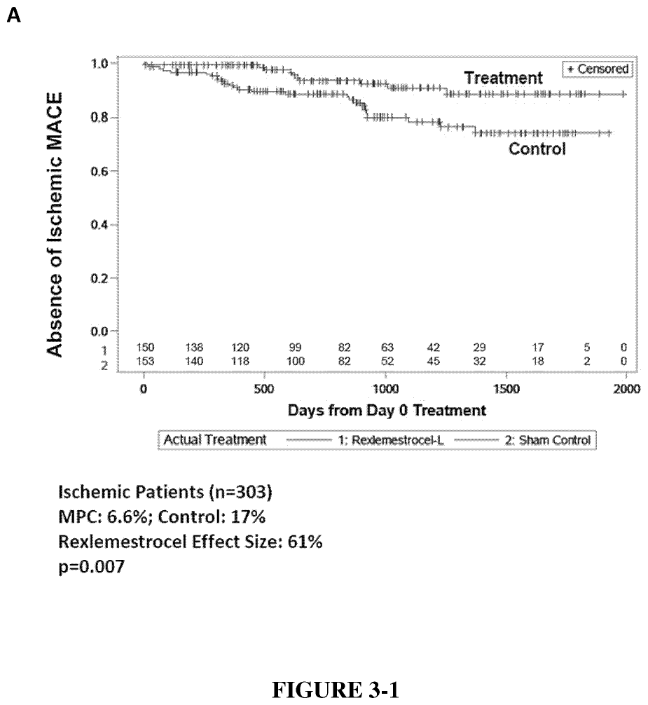

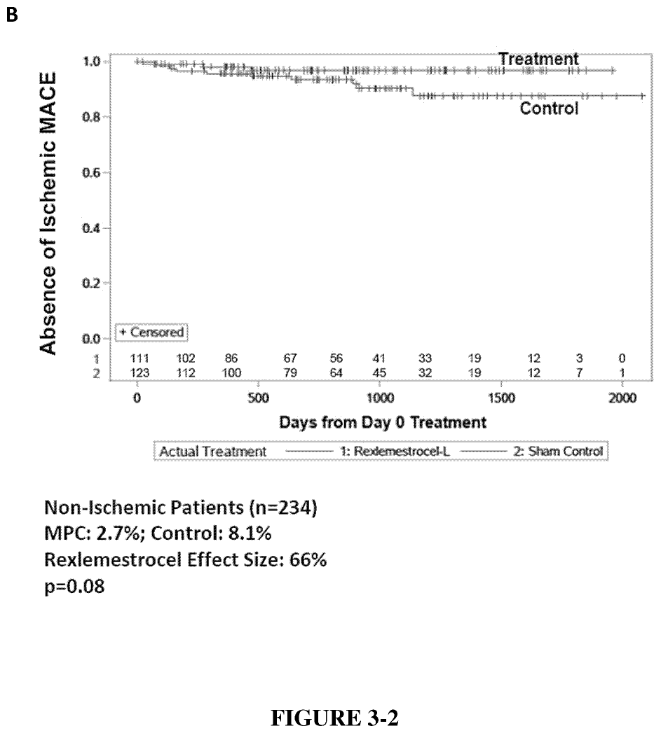

Cell therapy unexpectedly reduced incidence of ischemic MACE (MI, Stroke) by 60% relative to Controls (N=537 Patients; p=0.002; FIG. 1). FIG. 2 shows the significantly reduced incidence of ischemic MACE (MI, Stroke) observed relative to controls in NYHA Class II & III. These data suggest that cell therapy can reduce risk of ischemic events in patients with cardiomyopathy. FIG. 3 shows that the significantly reduced incidence of ischemic MACE (MI, Stroke) was observed in both ischemic and non-ischemic patients. Cardiomyopathy patients are at risk of occlusive plaque development due to ongoing inflammatory processes in these patients. These problematic processes appear to be inhibited by cell therapy in view of the general reduction of ischemic MACE observed in both ischemic and non-ischemic cardiomyopathy patients. Accordingly, the present data appears to underpin a general concept whereby cell therapy can be administered to reduce the risk of ischemic event(s) in patients suffering cardiomyopathy.

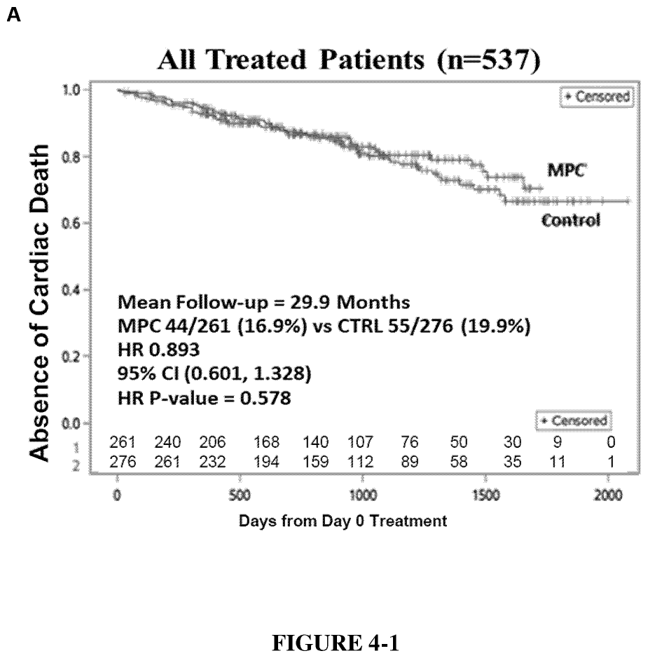

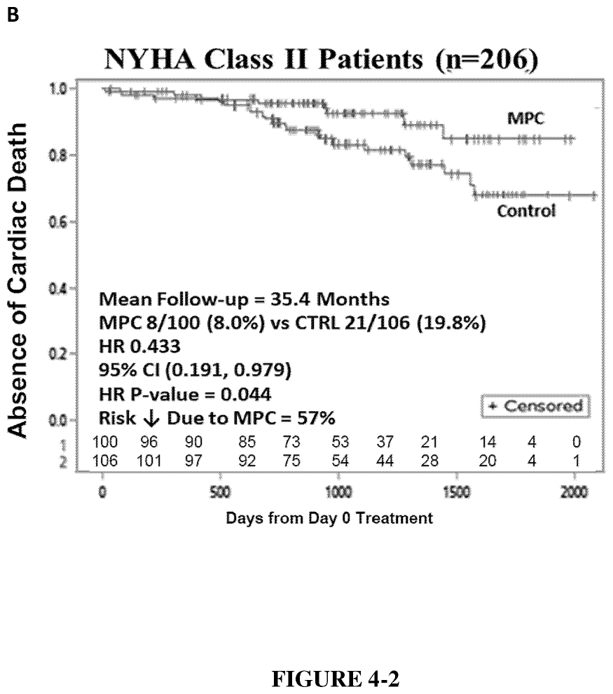

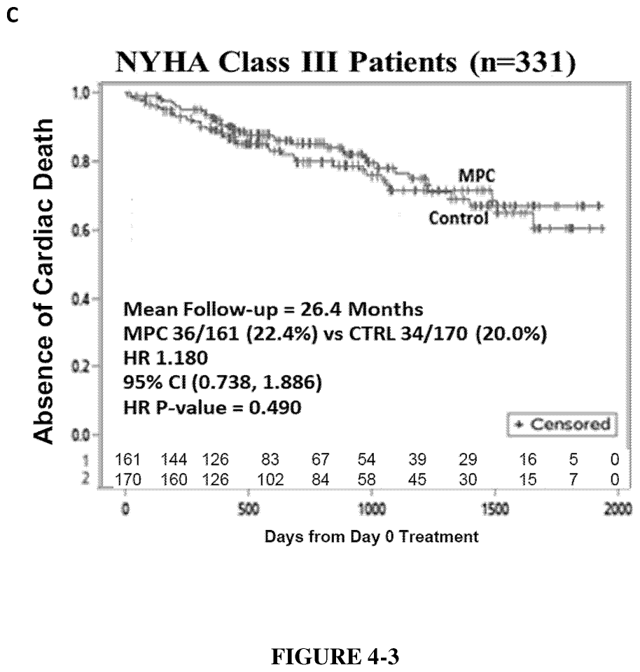

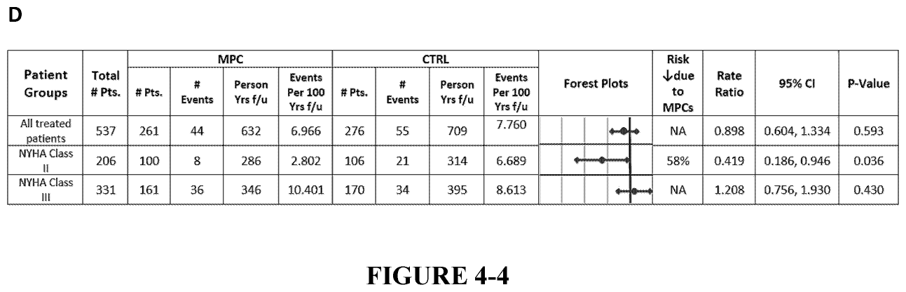

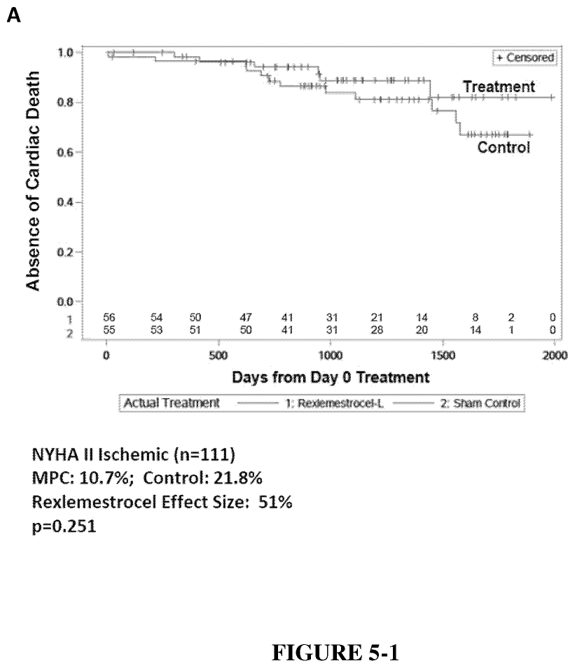

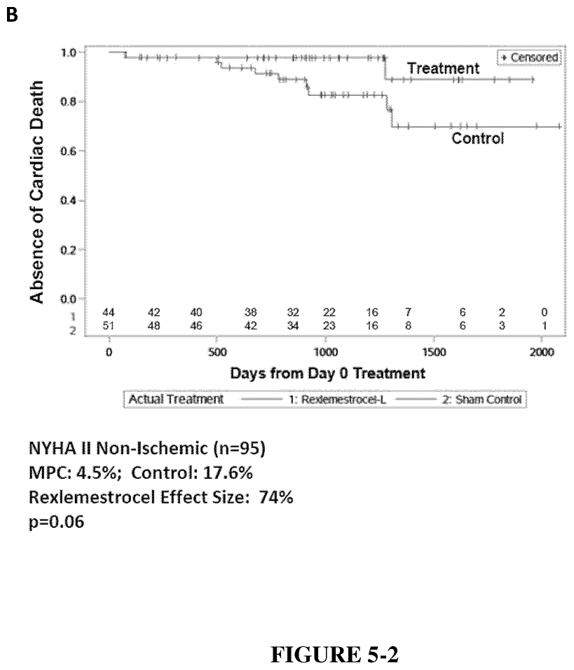

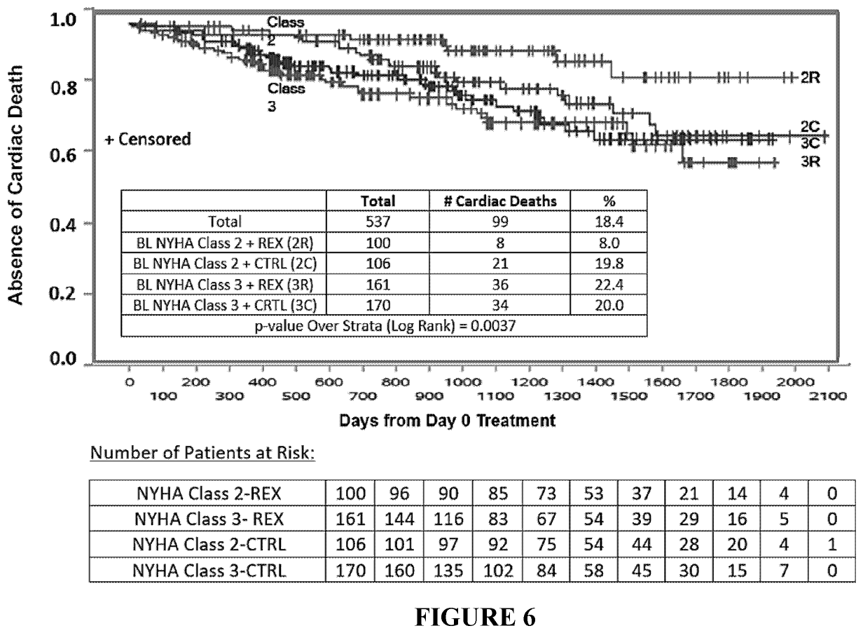

Surprisingly, cell therapy reduced cardiac death in NYHA Class II patients but did not reduce cardiac death in NYHA Class III patients (FIGS. 4 and 6). This result was surprising because it suggested a threshold level of viable myocardium was required to reduce cardiac death with cell therapy. In other words, patients with Class III heart failure may have progressed too far along the disease continuum for cell therapy to improve their survival. These findings suggest that cell therapy would be particularly effective in NYHA Class II patients. Given the capacity of the administered cells to induce new blood vessel formation in target tissue, the findings of the present inventors suggest a general concept for reducing cardiac death in patients graded lower than NYHA class III by administering cell therapy. As further support for this concept, the inventors noted that the observed reduction in cardiac death for NYHA Class II patients was maintained despite the cause of cardiomyopathy, with reductions in cardiac death being observed in ischemic and non-ischemic NYHA Class II patients (FIG. 5).

### 3) Improved Outcomes

### 3 Point MACE

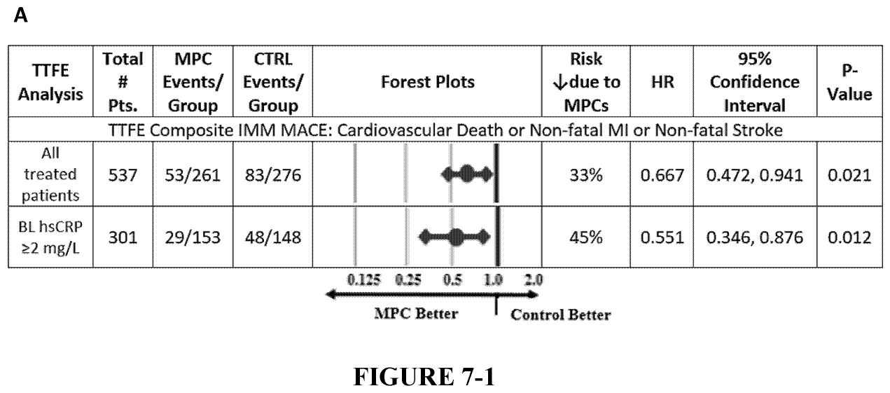

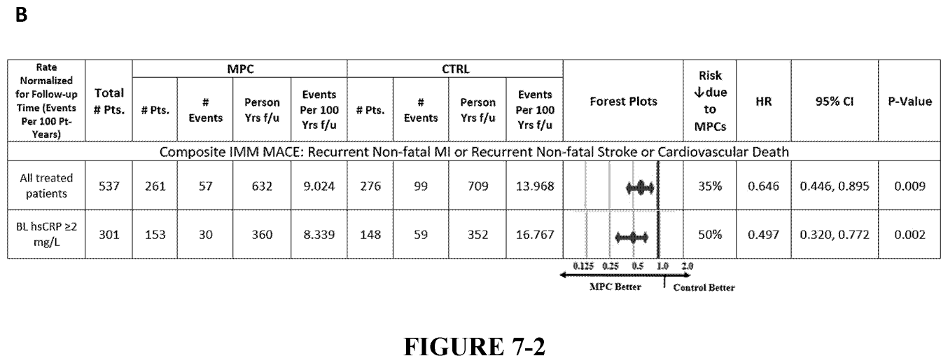

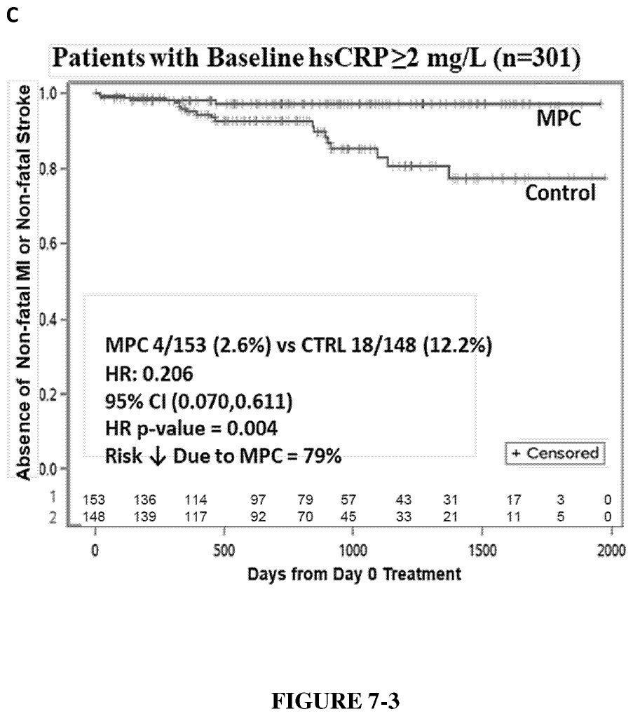

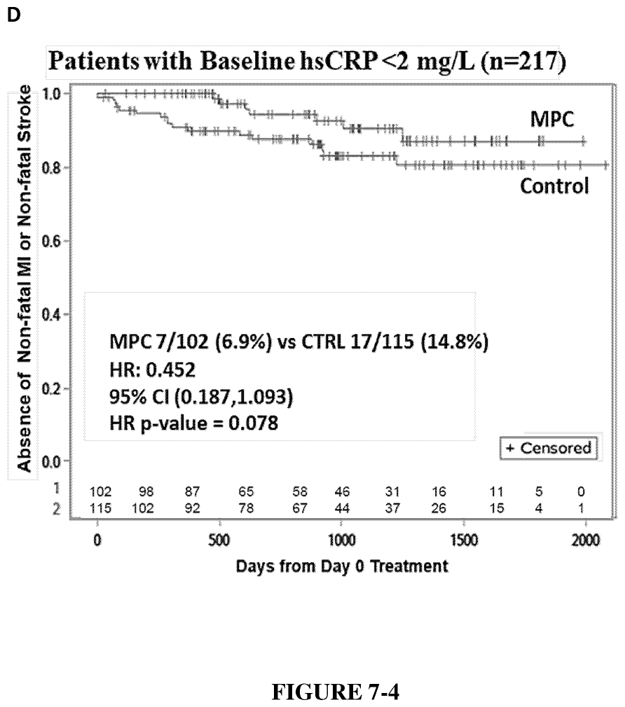

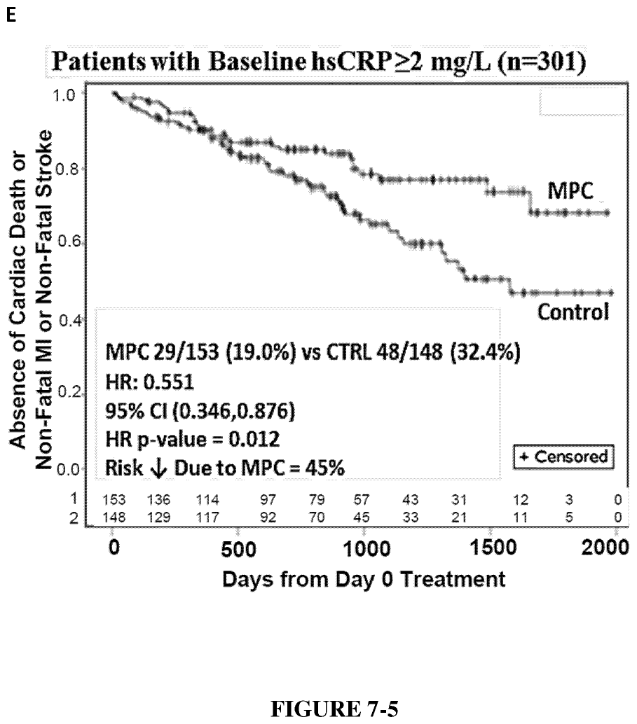

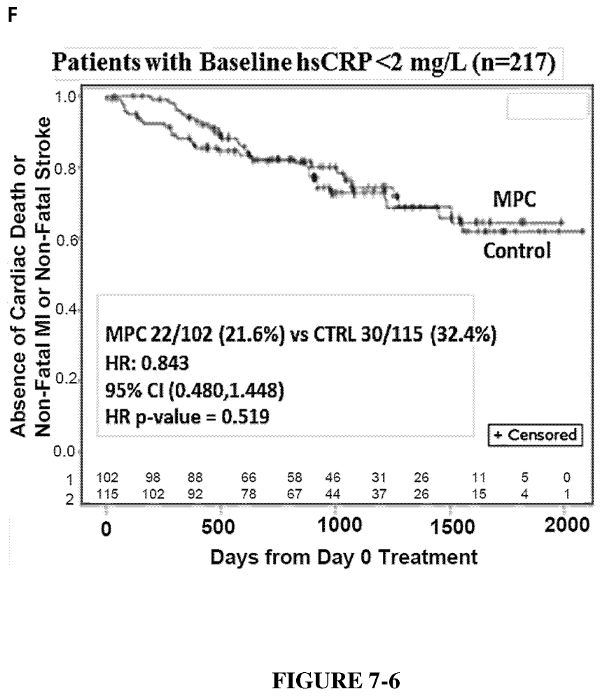

Subsequent analysis revealed that a single injection of cell therapy significantly decreased risk of the composite outcome of 3-point Irreversible Morbidity or Mortality MACE compared to controls across all 537 treated patients. Decreased risk was even more apparent in subject with CRP ≥2 mg/ml. Risk of 3-point MACE was reduced by 33% using a time to first event analysis [HR 0.667; 95% CI (0.472, 0.941); P=0.021; FIG. 7A] and by 35% in a recurrent event rate analysis normalized for time of follow-up (i.e., events per 100 patient-years) [HR 0.646; 95% CI (0.466, 0.895); P=0.009; FIG. 7B]. Kaplan-Meier curves for this composite outcome in patients with plasma hsCRP levels ≥2 mg/L or <2 mg/L are depicted in FIG. 7C. As shown in FIG. 7C, in all treated patients with CRP ≥2 mg/L, cell therapy significantly reduced the risk of:
Non-fatal MI and non-fatal stroke (FIG. 7C1); and,
Composite of cardiac death or ischemic MACE (MI or Stroke) (FIG. 7C2).

### Ischemic MACE

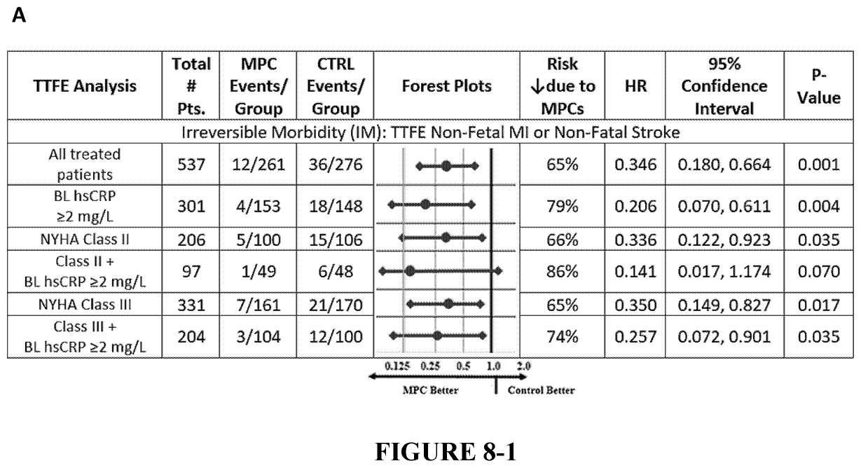

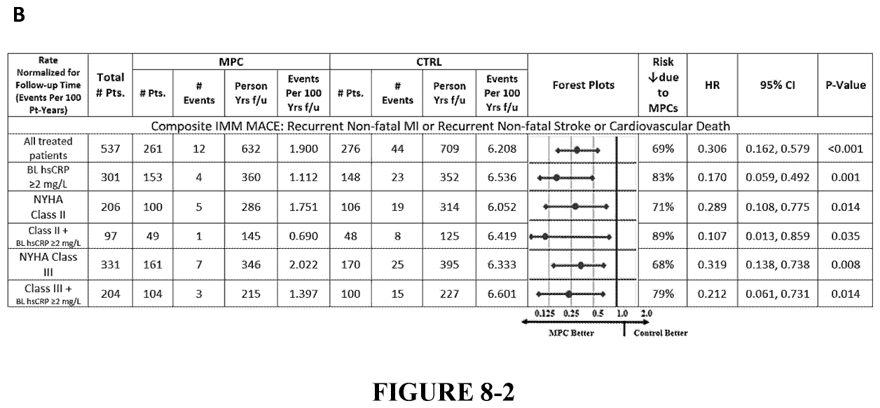

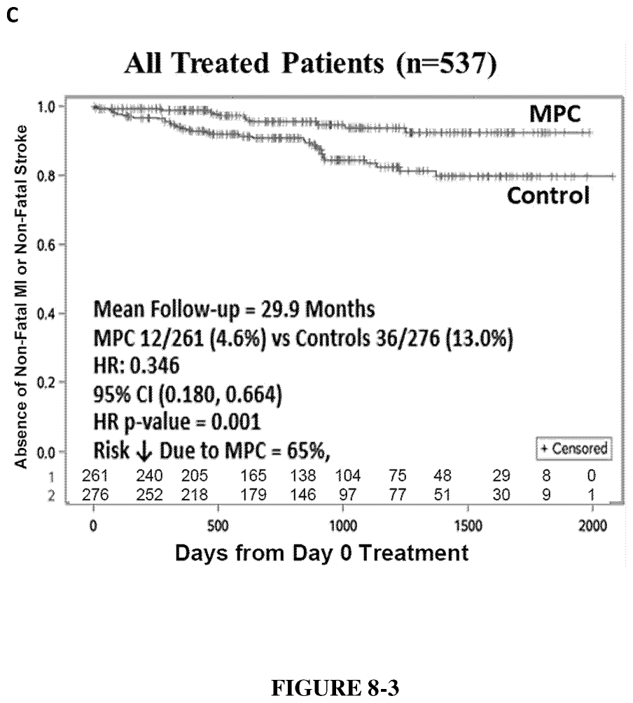

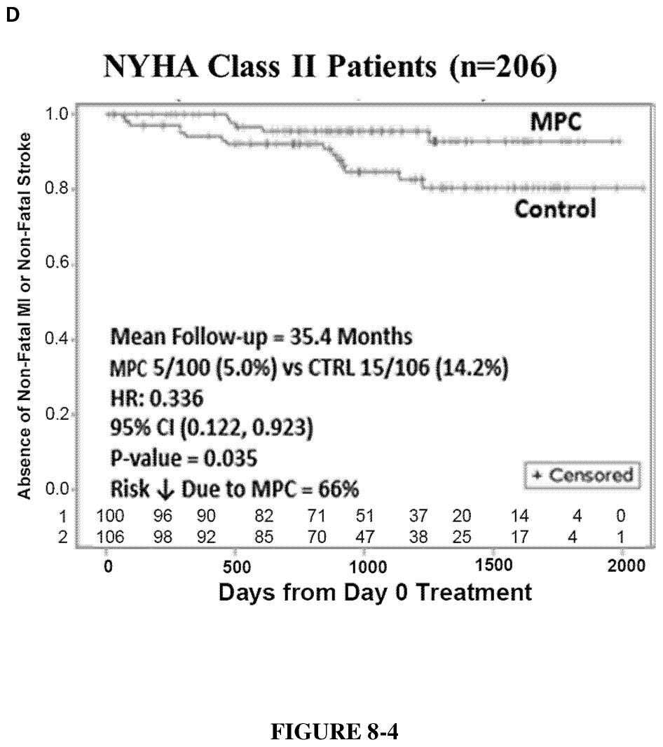

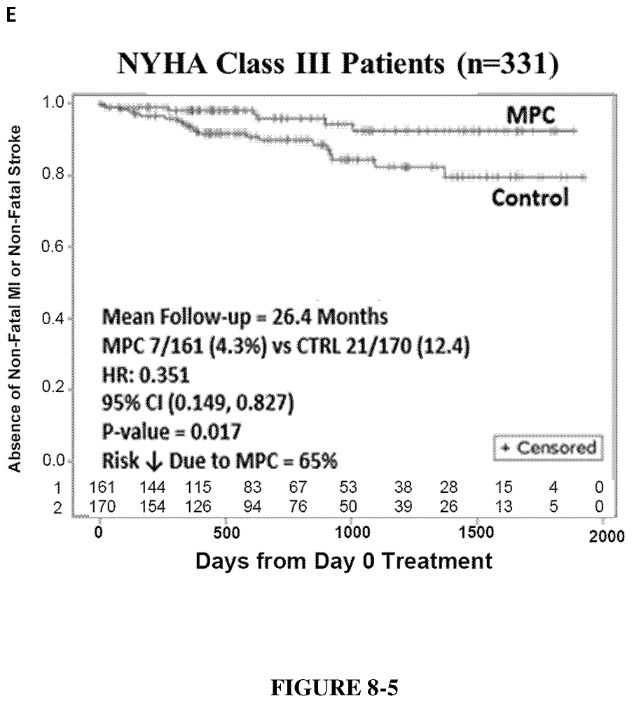

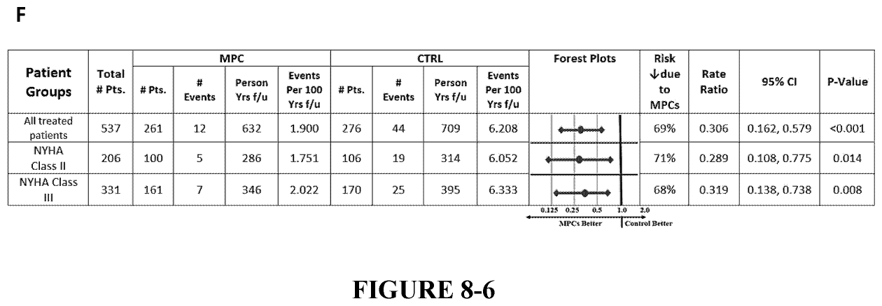

Across all 537 treated patients, a single injection of cell therapy decreased risk for occurrence of Irreversible Morbidity (non-fatal MI or non-fatal stroke) by 65% compared to controls using time to first event analysis (TTFE) [HR 0.346; 95% CI (0.180, 0.664); P=0.001; FIG. 8A] and by 69% using recurrent event rate normalization [HR 0.306; 95% CI (0.162, 0.579); P<0.001; FIG. 8B].

Pre-specified sub-group analyses were performed for all treated patients based on presence or absence of inflammation at the time of treatment. A single injection of cell therapy in the 301 patients with inflammation (CRP ≥2 mg/L) decreased risk of non-fatal MI or non-fatal stroke by 79% using TTFE [HR 0.206; 95% CI (0.070, 0.611); P=0.004; FIG. 8A] and by 83% using recurrent event rate normalization [HR 0.170; 95% CI (0.059, 0.492); P=0.001; FIG. 8B]. Taken together with the above referenced 3 point-MACE analysis, these data underpin a rationale for selecting and treating patients heart failure an active inflammation, preferably as defined by CRP ≥2 mg/L.

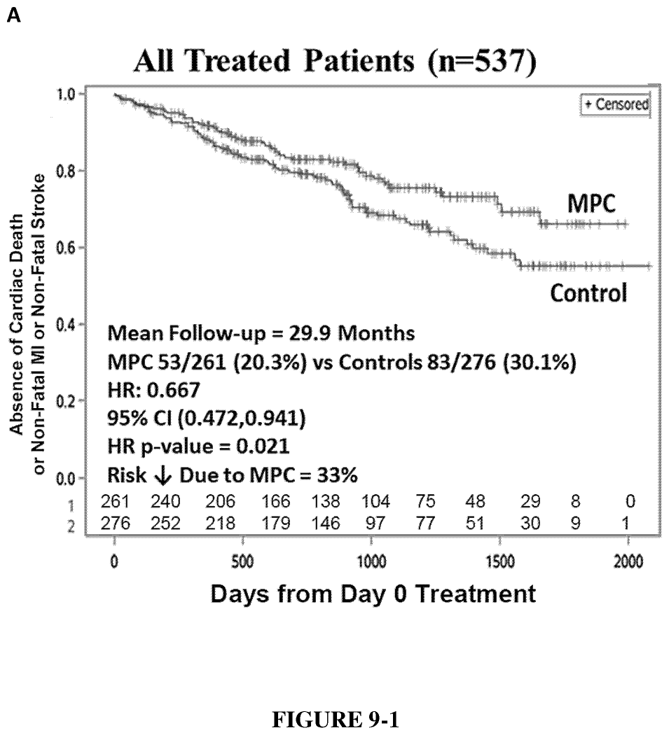

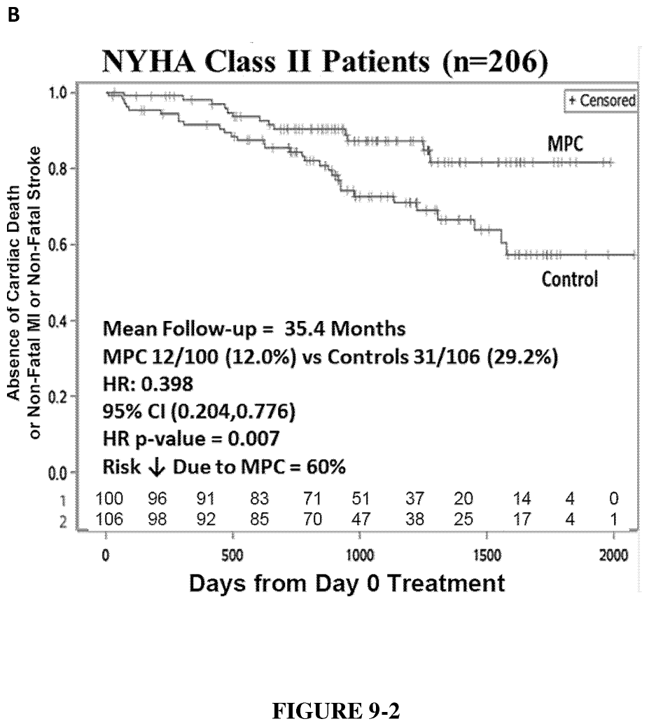

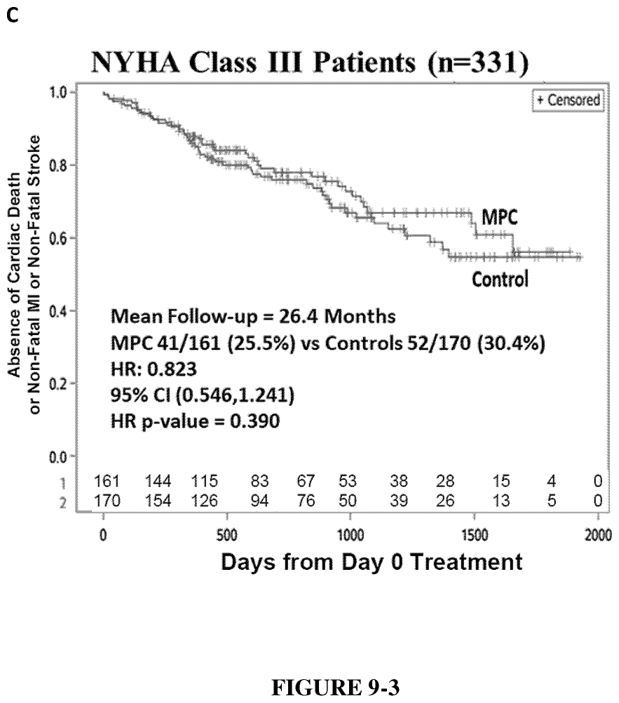

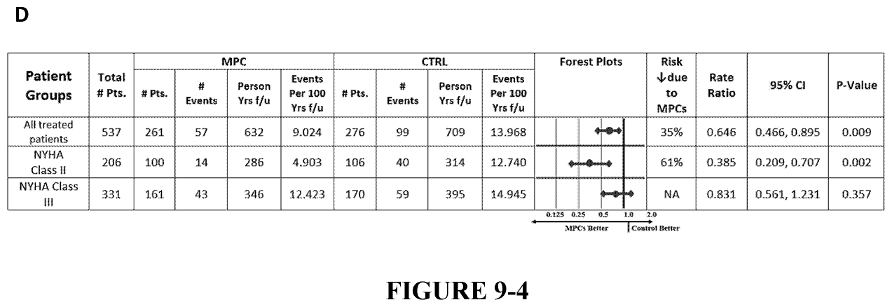

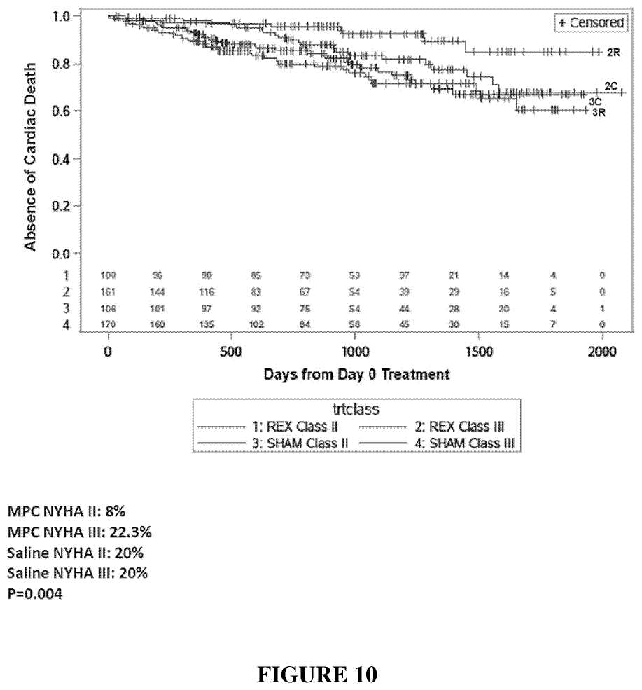

Cell therapy significantly reduced composite of cardiac death or ischemic MACE (MI or Stroke) in all patients by 33% (FIG. 9). Further analysis of patient groups then revealed that cell therapy significantly reduced by 60% composite of cardiac death or ischemic MACE (MI or Stroke) in NYHA Class II patients compared to controls, further supporting the rationale of selecting and treating patients with NYHA Class II heart failure (FIG. 9). It was also noted that cell therapy prevented progression of cardiac death in NYHA Class II patients over multiple years of follow-up (FIG. 10).

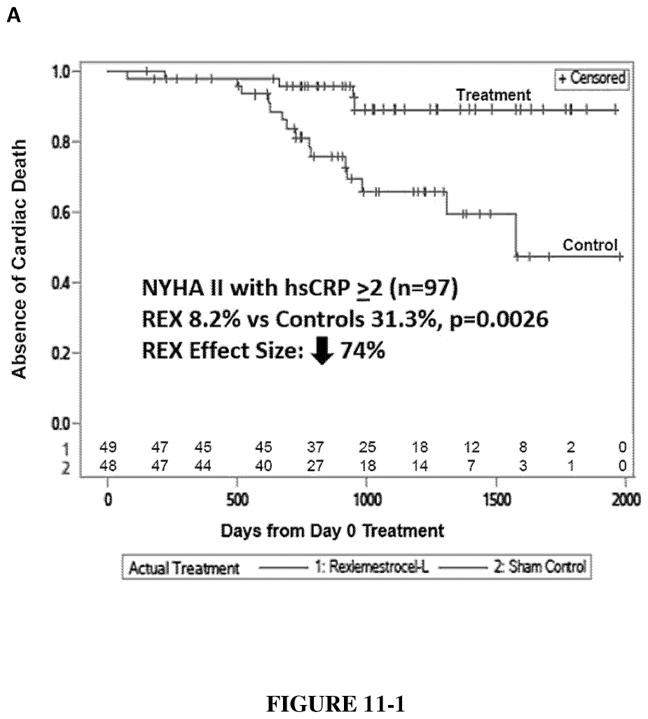

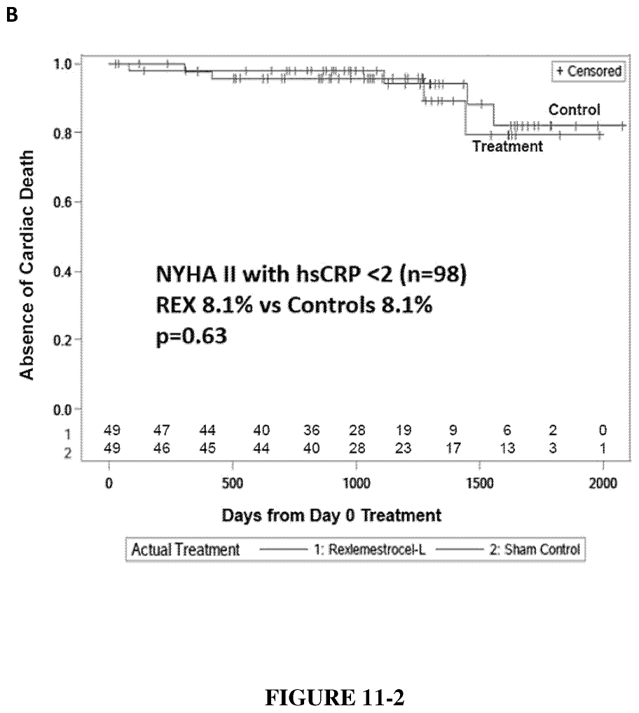

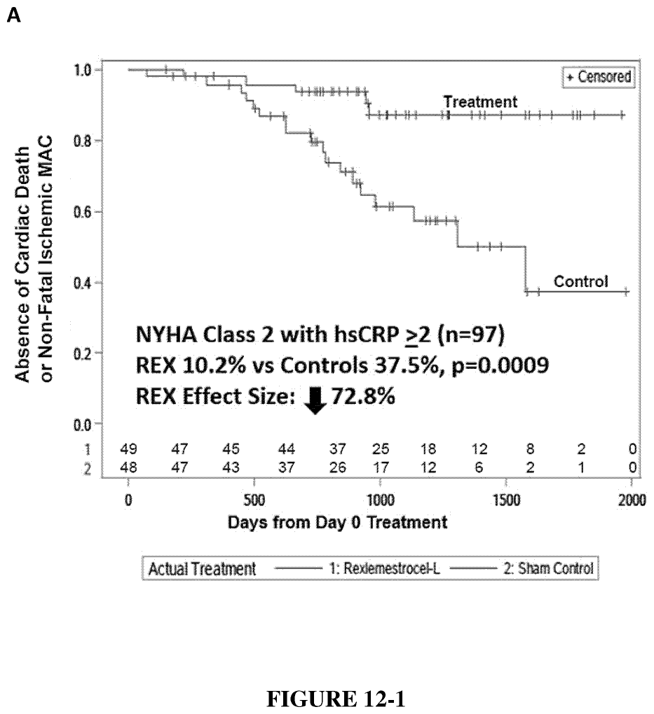

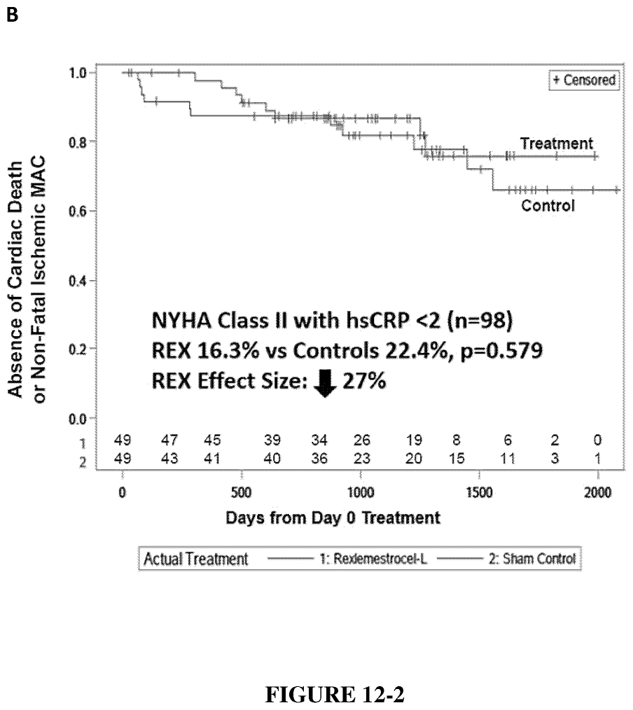

Cell therapy also unexpectedly reduced risk of cardiac death (FIG. 11) and 3-point MACE (Cardiac death/MI/Stroke; FIG. 12) in NYHA Class II patients with elevated CRP levels, in particular, CRP levels ≥2 mg/L. These beneficial effects were not evident in patients with baseline CRP <2 mg/, suggesting that cell therapy may be particularly beneficial in the presence of active inflammation.

Further data analysis revealed that CRP was an important marker of cardiac death. As shown in FIG. 11, patients having elevated CRP levels (≥2 mg/L) had significantly increased risk of cardiac death. These data further support the treatment of NYHA class II patients with cell therapy, in particular when those patients have elevated CRP levels, for example CRP ≥2 mg/L. These data also indicate the utility of CRP level as an indicator or patients at risk of cardiac death.

Myocardial Ischemia and/or Diabetes

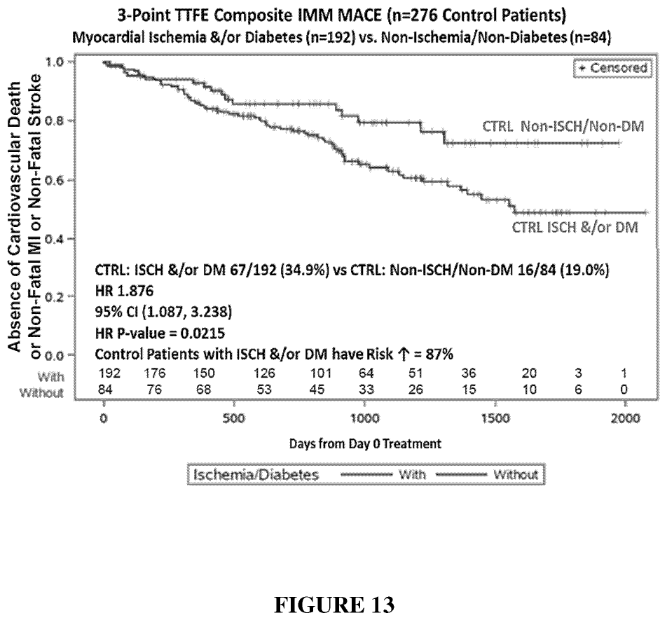

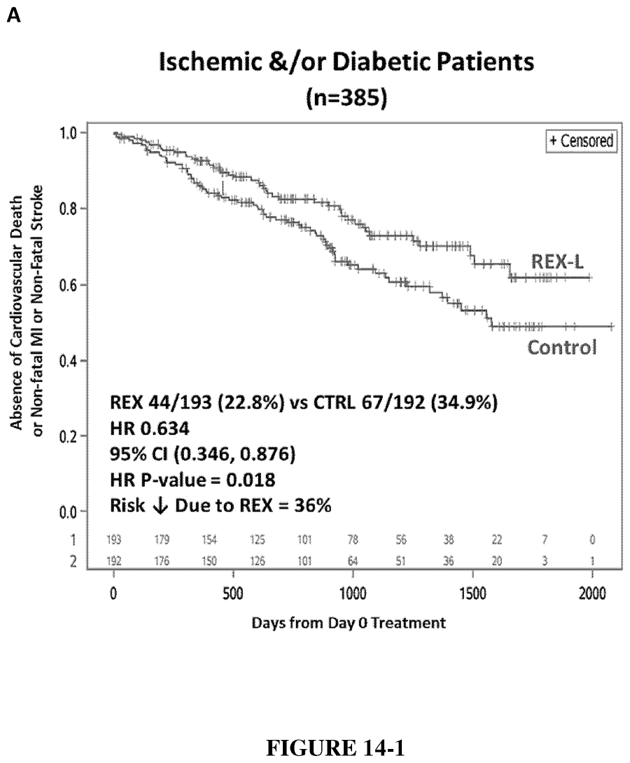

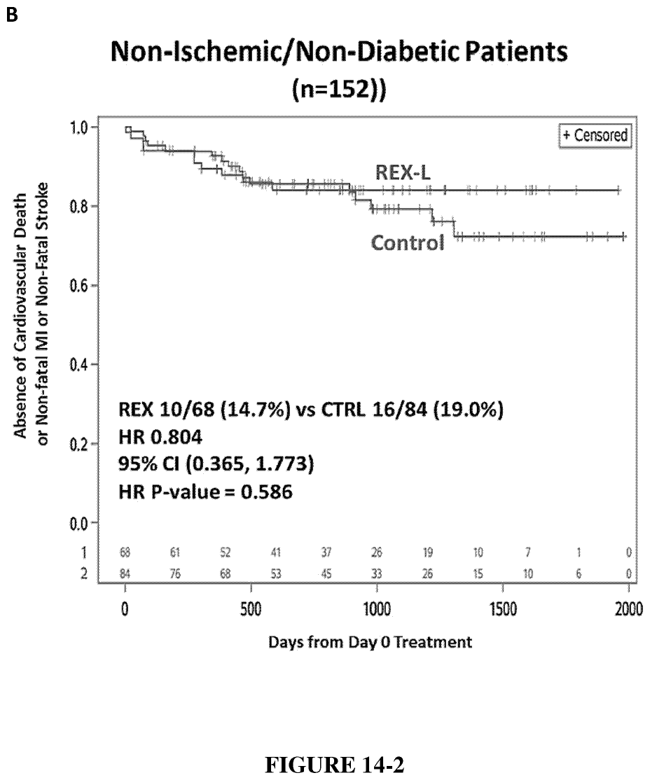

HF patients with micro- or macro-vascular disease (myocardial ischemia or diabetes) represent some of the most difficult to treat patients in view of their poor outcomes (highest hazard ratio of treated patients (Table 1)). Indeed, analysis of the present patient population revealed that untreated control heart failure patients with myocardial ischemia and/or diabetes (n=276) are associated with significantly worse outcomes, as measured by 3-Point IMM MACE, than other heart failure control patients (n=84). In particular, untreated control heart failure patients with myocardial ischemia and/or diabetes had a significantly increased incidence of cardiovascular death or non-fatal MI or non-fatal stroke (FIG. 13). However, cell therapy was surprisingly effective in these patients with myocardial ischemia and/or diabetes (n=385; Table 1), reducing the risk of 3-point composite IMM MACE by 36% (FIG. 14). These data suggest that cell therapy is particularly beneficial in HF patients with micro- or macro-vascular disease, in particular myocardial ischemia and/or diabetes, underpinning a rationale for selecting these patients for treatment.

TABLE 1
Evaluation of cell therapy on 3-Point IMM MACE
In pre-specified patient populations with
reproducibly high-risk for poor outcomes.

| | | | 95% | |
|---|---|---|---|---|
| | Number of | Hazard | Confidence | |
| | Patients | Ratio | Interval | P-value |
| Myocardial Ischemic | 385 | 0.63 | 0.434, 0.928 | 0.119 |
| &/or Diabetes | | | | |
| Myocardial Ischemia¹ | 3.3 | 0.66 | 0.436, 0.988 | 0.044 |
| Diabetes² | 231 | 0.66 | 0.400, 1.094 | 0.108 |
| HF hospitalization | 330 | 0.66 | 0.414, 1.048 | 0.078 |
| within prior 1-9 months | | | | |
| Prior Myocardial | 280 | 0.70 | 0.452, 1.069 | 0.098 |
| Infarction | | | | |
| NT-proBNP > 1000 | 365 | 0.74 | 0.505, 1.083 | 0.121 |
| LVESV > 100 mL | 431 | 0.75 | 0.517, 1.073 | 0.114 |
| LVEF < 30% | 299 | 0.77 | 0.508, 1.116 | 0.206 |

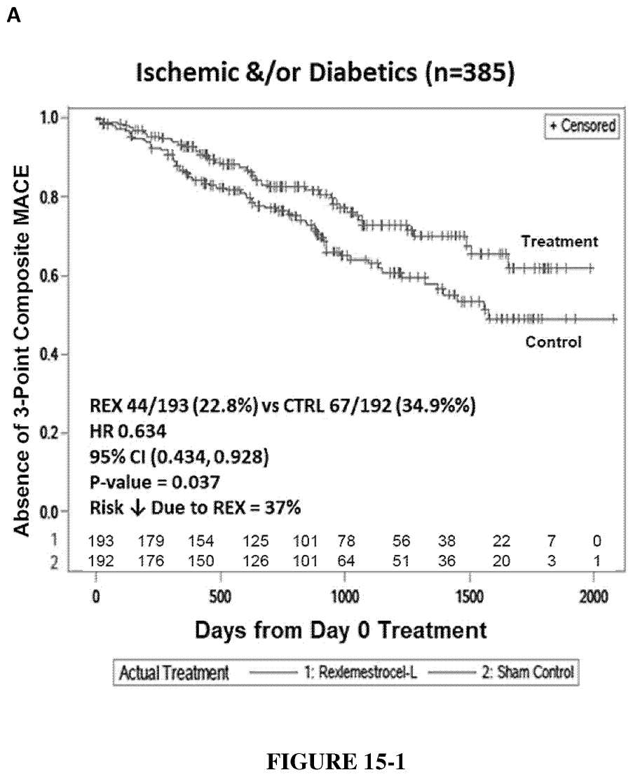

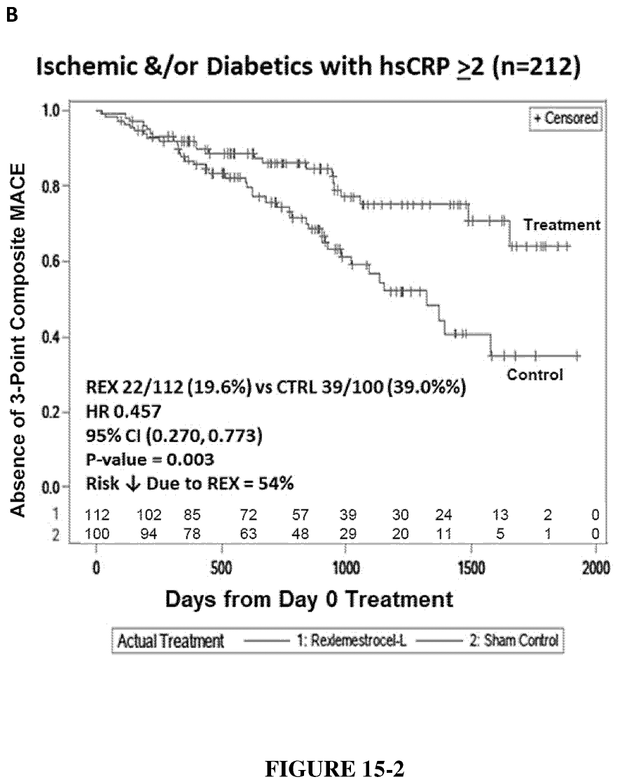

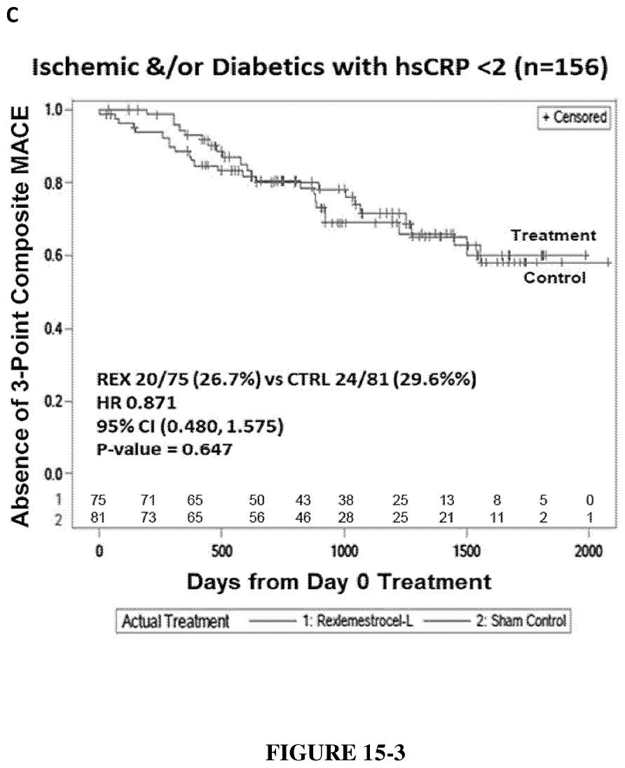

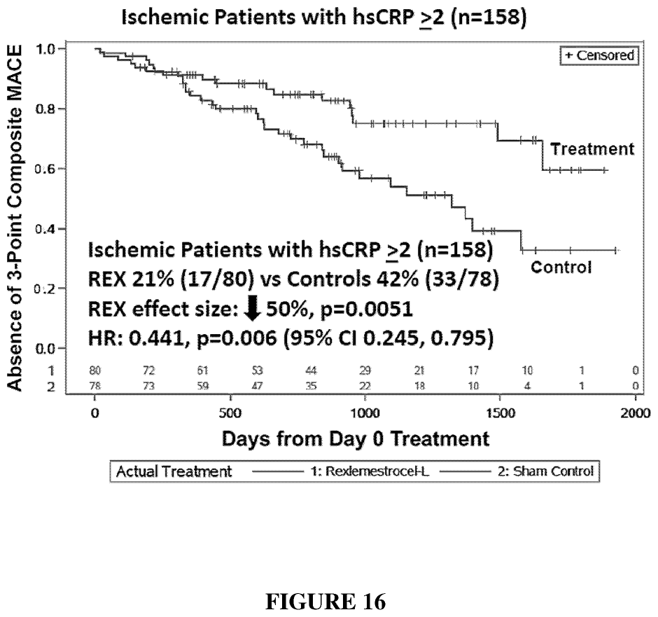

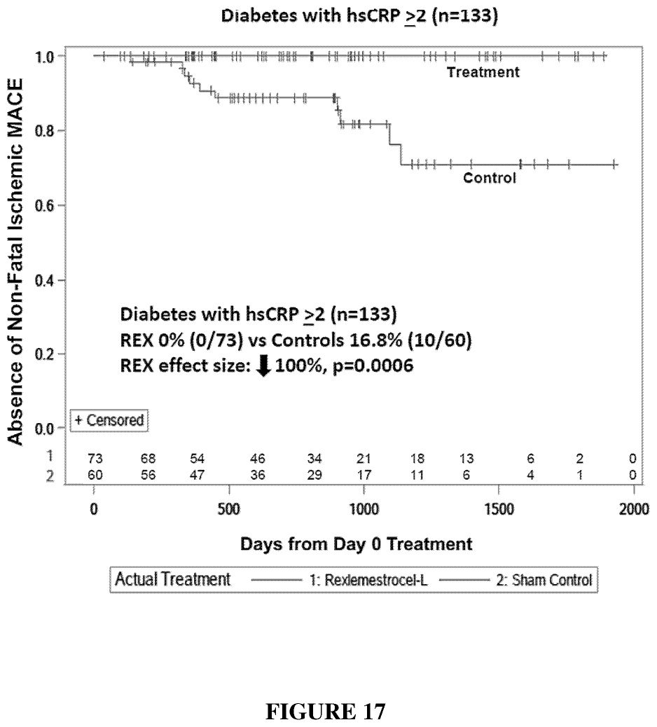

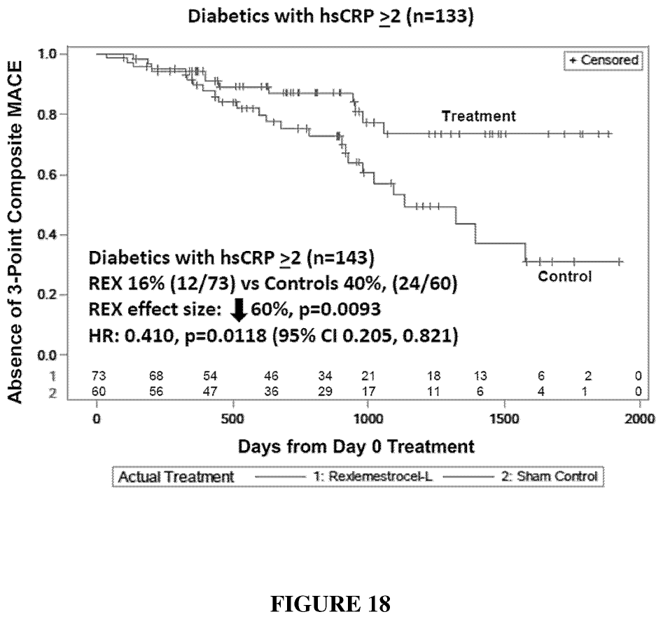

As shown in FIG. 15, the above referenced therapeutic efficacy was even more pronounced in HF patients with active inflammation (i.e. CRP levels ≥2 mg/L) in addition to micro-vascular disease (myocardial ischemia; FIG. 16) or macro-vascular disease (diabetes; FIG. 17 and FIG. 18). These data provide further support for therapeutic efficacy of cell therapy in patients with active inflammation. Furthermore, they suggest that cell therapy is particularly beneficial in HF patients with active inflammation and either micro- or macro-vascular disease, in particular myocardial ischemia or diabetes, underpinning a rationale for also selecting these patients for treatment.

### Comparative Data

Heart failure with reduced ejection fraction (HFrEF) is a progressive disease that leads to recurrent hospitalizations and increased risk of death. Treatments that reduce irreversible morbidity (non-fatal myocardial infarction (MI) or non-fatal stroke) and mortality remain elusive. It is therefore particularly surprising that cell therapy outperformed other investigational agents evaluated for cardiovascular risk reduction using 3-Point IMM MACE (TTFE composite for cardiovascular death or non-fatal MI or non-fatal stroke) (Table 2).

TABLE 2
Investigational agents evaluated for cardiovascular risk reduction using 3-Point IMM MACE:
Comparison with cell therapy in patients with myocardial ischemia &/or diabetes.

| | | | | | | | # |
|---|---|---|---|---|---|---|---|
| | | | Hazard | Risk | | | Randomized |
| Medication | Drug Class | Clinical Trial | Ratio | Reduction | 95% CI | P-value | Patients |
| Liraglutide | GLP-1 | LEADER | 0.87 | 13% | 0.78, 0.97 | 0.01 | 9,340 |
| | Receptor | | | | | | |
| | Agonist (RA) | Heart Failure | 0.94 | 6% | 0.72, 1.21 | — | 1,305 |
| | | Sub-group | | | | | |
| Dulaglutide | GLP-1 | REWIND | 0.88 | 12% | 0.79, 0.99 | 0.03 | 9,901 |
| | Receptor | | | | | | |
| | Agonist (RA) | | | | | | |
| Empagliflozin | SGLT-2 | EMPA-REG | 0.86 | 14% | 0.74, 0.99 | 0.04 | 7,020 |
| | Inhibitor | | | | | | |
| Canagliflozin | SGLT-2 | CANVAS + | 0.86 | 14% | 0.75, 0.97 | 0.02 | 10,142 |
| | Inhibitor | CANVAS-R | | | | | |
| | | Heart Failure | 0.80 | 20% | 0.61, 1.05 | — | 1,461 |
| | | Sub-group | | | | | |
| Dapagliflozin | SGLT-2 | DECLARE Timi | 0.93 | 7% | 0.84, 1.03 | — | 17,160 |
| | Inhibitor | 58 | | | | | |
| | | Heart Failure | 1.01 | 0% | 0.81, 1.27 | — | 1,724 |
| | | Sub-group | | | | | |
| Ertugliflozin | SGLT-2 | VERTIS CV | 0.99 | 1% | 0.88, 1.12 | — | 8,246 |
| | Inhibitor | | | | | | |
| Rexlemestrocel-L | Mesenchymal | DREAM HF | 0.63 | 37% | 0.43, 0.93 | 0.037 | 385 |
| | Precursor Cells | Ishcmics &/or | | | | | |
| | | Diabetes | | | | | |
| | | Ischemics &/or | 0.46 | 54% | 0.24, 0.77 | 0.003 | 212 |
| | | Diabetes With | | | | | |
| | | Baseline | | | | | |
| | | hsCRP ≥ 2 mg/L | | | | | |

1. A method for treating or preventing progressive heart failure in a subject, the method comprising administering to the subject a composition comprising mesenchymal lineage precursor or stem cells, wherein the subject's CRP level is ≥2 mg/L and, wherein the subject has micro-vascular disease and/or macro-vascular disease.

2. A method of reducing risk of cardiac death, or a non-fatal ischemic event in a subject, the method comprising administering to the subject a composition comprising mesenchymal lineage precursor or stem cells, wherein the subject has micro-vascular disease and/or macro-vascular disease, and wherein the subject's CRP level is ≥2 mg/L.

3. A method of selecting heart failure patients for treatment with cell therapy, the method comprising i) assessing the subject for a micro-vascular disease and/or a macro-vascular disease and CRP level, and ii) selecting a subject having a micro-vascular disease and/or a macro-vascular disease and CRP levels of ≥2 mg/L for treatment, preferably, wherein the treatment comprises administering a composition comprising mesenchymal lineage precursor or stem cells.

4. The method according to claim 1, wherein the subject's progressive heart failure is due to ischemic cardiomyopathy.

5. (canceled)

6. The method according to claim 1, wherein;
the subject has a LVEF of less than about 45%;
the subject has a LVEF of less than 40%;
the subject has a LVESV greater than 70 ml; or
the subject has a LVESV between 70 ml and 160 ml.

7.-8. (canceled)

9. The method according to claim 1, wherein the subject has Class II or Class III heart failure according to the New York Heart Association (NYHA) classification scale.

10. (canceled)

11. The method according to claim 1, wherein;
the subject's level of N-terminal pro-B-type natriuretic peptide (NT-proBNP) is:
>1000 μg/mL, or,
between 1000 pg/ml and 2500 pg/ml;
the subject's C-reactive protein (CRP) level is <5 mg/L, <4 mg/L, or <3 mg/L;
the subject has had a heart failure hospitalisation event over the previous 9 months;
the subject has persistent left ventricular dysfunction; or
the subject's heart failure results from an ischaemic event or from a non-ischaemic event.

12.-15. (canceled)

16. The method according to claim 1, wherein the subject has a reduced risk of cardiac death after treatment, wherein the reduced risk is relative to risk of cardiac death in a subject that has not been administered mesenchymal lineage precursor or stem cells: or
wherein the subject has a reduced risk of ischaemic MACE after treatment, wherein the MACE is MI or stroke.

17.-18. (canceled)

19. The method according to claim 1, wherein the composition is administered transendocardially and/or intravenously.

20. The method according to claim 1, wherein the mesenchymal lineage precursor or stem cells are mesenchymal precursor cells (MPCs) or wherein the mesenchymal lineage precursor or stem cells are mesenchymal stem cells (MSCs).

21.-22. (canceled)

23. The method according to claim 1, wherein;
the cells are allogeneic;
the cells are culture expanded;
the cells are isolated from bone mononuclear cells with an anti-STRO-3 antibody;
the cells are STRO-3+ before they are culture expanded; and/or
the cells have been cryopreserved.

24.-26. (canceled)

27. The method according to claim 1 which comprises administering between 1×10² and 2×10⁸ cells.

28. (canceled)

29. The method according to claim 1, wherein the composition further comprises Plasma-Lyte A (70%), DMSO (10%), HSA (25%) solution, the HSA solution comprising 5% HSA and 15% buffer, wherein the composition comprises greater than 6.68×10⁶ viable cells/m.

30. (canceled)

31. The method according to claim 1, wherein the composition comprises human bone marrow-derived allogeneic mesenchymal precursor cells (MPCs) isolated from bone mononuclear cells with anti-STRO-3 antibodies, expanded ex vivo, and cryopreserved.

32.-34. (canceled)

35. The method according to claim 32, wherein the ischemic event:
is formation of an arterial occlusion;
a cerebrovascular occlusion;
a cardiac occlusion;
stroke; or
myocardial infarction.

36.-40. (canceled)

41. The method according to claim 1, wherein the micro-vascular disease is myocardial ischemia.

42. The method according to claim 1, wherein the macro-vascular disease is diabetes.

43. The method according to claim 1, wherein the subject's progressive heart failure is due to non-ischemic cardiomyopathy.

44. The method according to claim 43, wherein the non-ischemic cardiomyopathy is diabetic cardiomyopathy.

45. The method according to claim 1, wherein the subject's progressive heart failure is heart failure with reduced ejection fraction (HFrEF).
